(19) United States
(12) Patent Application Publication
(10) Pub. No.: US 2023/0114905 A1
(43) Pub. Date: Apr. 13, 2023

(54) HIGHLY MULTIPLEXABLE ANALYSIS OF PROTEINS AND PROTEOMES

(71) Applicant: NAUTILUS BIOTECHNOLOGY, INC., Seattle, WA (US)

(72) Inventors: Jarrett D. EGERTSON, Rancho Palos Verdes, CA (US); James SHERMAN, Kingston, WA (US); Vadim LOBANOV, Seattle, WA (US); Parag MALLICK, San Mateo, CA (US); Ellis ANDERSON, Seattle, WA (US)

(21) Appl. No.: 18/045,036

(22) Filed: Oct. 7, 2022

Related U.S. Application Data
(60) Provisional application No. 63/254,420, filed on Oct. 11, 2021.

(51) Int. Cl. G01N 33/68; C40B 30/04

(52) U.S. Cl. CPC G01N 33/6803; C40B 30/04

(57) ABSTRACT

A method of identifying an extant protein, including (a) providing inputs including: (i) a binding profile, wherein the binding profile includes a plurality of binding outcomes for binding of the extant protein to a plurality of different affinity reagents, wherein individual binding outcomes of the plurality of binding outcomes include a measure of binding between the extant protein and a different affinity reagent of the plurality of different affinity reagents, (ii) a database including information characterizing or identifying a plurality of candidate proteins, and (iii) a binding model; (b) determining a probability for each of the affinity reagents binding to each of the candidate proteins in the database according to the binding model; and (c) identifying the extant protein as a selected candidate protein having a probability for binding each of the affinity reagents that is most compatible with the binding profile for the extant protein.

### CROSS-REFERENCE TO RELATED APPLICATIONS

This application claims priority to U.S. Provisional Application No. 63/254,420, filed on Oct. 11, 2021, which application is incorporated herein by reference in its entirety.

### FIELD

Some embodiments relate to a method of performing a protein binding assay. More particularly, some embodiments relate to a method of performing a protein binding assay to identify extant proteins by using a binding profile which includes a plurality of binding outcomes for binding of the extant protein to a plurality of different affinity reagents.

### BACKGROUND

The proteome is among the most dynamic and valuable sources of biological insight. Current proteomics techniques are limited in their sensitivity and throughput, covering at best 35% of the human proteome in a single experiment (see Blume et al., Nat Commun 11, 3662 (2020) and Clark et al., Cell 180, 207 (2020), each of which is incorporated herein by reference). Despite the wealth of insights gained from now routine genomics and transcriptomics studies in biomedical research, a large gap remains between genome/transcriptome and phenotype. Proteomics is crucial to bridging this gap as proteins constitute the main structural and functional components of cells. However, protein sequencing technologies lag behind DNA sequencing technologies, in part due to the complex nature of proteins and proteomes as well as the high dynamic range (˜10⁹) in the quantities of different proteins present at any given time in any given cell (see Aebersold et al., Nat Chem Biol 14, 206-214 (2018), which is incorporated herein by reference). Moreover, about 10% of the proteins predicted to comprise the human proteome have not been confidently observed at all (see Omenn et al., J Proteome Res 19, 4735-4746 (2020) and Adhikari et al., Nat Commun 11, 5301 (2020), each of which is incorporated herein by reference).

Recently, single-molecule identification has been postulated as a method to analyze small samples (including single cells) and rare proteins (see Alfaro et al., Nat Methods 18, 604-617 (2021) and Restrepo-Perez et al., Nat Nanotechnol 13, 786-796 (2018), each of which is incorporated herein by reference). Traditional bulk identification techniques like mass spectrometry and immunoassays have been adapted towards detection of single proteins (see Keifer & Jarrold, Mass Spectrom Rev 36, 715-733 (2017) and Risin et al., Nat Biotechnol 28, 595-599 (2010), each of which is incorporated herein by reference). Several concepts have been proposed to achieve single-molecule protein sequencing. These all use sequential processes to determine the positional information of amino acids within proteins e.g., Edman-type degradation (Swaminathan, et al. Nat Biotechnol (2018) and Swaminathan, et al., PLoS Comput Biol 11, e1004080 (2015), each of which is incorporated herein by reference) or directional protein translocation through a nanopore channel (Kolmogorov, et al., PLoS Comput Biol 13, e1005356 (2017), each of which is incorporated herein by reference). However, no current method achieves both single-molecule sensitivity and high throughput at a level that is commensurate with the complexity of the human proteome. Thus, there exists a need for comprehensive proteome analysis. The present disclosure satisfies this need and provides other advantages as well.

### SUMMARY

The present disclosure provides a method of identifying an extant protein. The method can include steps of (a) providing inputs to a computer processor, the inputs including: (i) a binding profile, wherein the binding profile includes a plurality of binding outcomes for binding of the extant protein to a plurality of different affinity reagents, wherein individual binding outcomes of the plurality of binding outcomes include a measure of binding between the extant protein and a different affinity reagent of the plurality of different affinity reagents, the binding profile including positive binding outcomes and negative binding outcomes, (ii) a database including information characterizing or identifying a plurality of candidate proteins, and (iii) a binding model for each of the different affinity reagents; (b) determining a probability for each of the affinity reagents binding to each of the candidate proteins in the database according to the binding model; and (c) identifying the extant protein as a selected candidate protein, the selected candidate protein being a candidate protein in the database having a probability for binding each of the affinity reagents that is most compatible with the binding profile for the extant protein. Optionally, the inputs can further include (iv) a non-specific binding rate including a probability of a non-specific binding event occurring for one or more of the different affinity reagents.

Also provided is method of identifying an extant protein, which includes steps of: (a) contacting a plurality of different affinity reagents with a plurality of extant proteins in a sample; (b) acquiring binding data from step (a), wherein the binding data includes a plurality of binding profiles, wherein each of the binding profiles includes a plurality of binding outcomes for binding of an extant protein of step (a) to a plurality of different affinity reagents, wherein individual binding outcomes of the plurality of binding outcomes include a measure of binding between an extant protein of step (a) and a different affinity reagent of the plurality of different affinity reagents, each of the binding profiles including positive binding outcomes and negative binding outcomes; (c) providing a database including information characterizing or identifying a plurality of candidate proteins; (d) providing a binding model for each of the different affinity reagents; (e) determining a probability for each of the affinity reagents binding to each of the candidate proteins in the database according to the binding model; and (f) identifying the extant proteins as selected candidate proteins, the selected candidate proteins being candidate proteins in the database having a probability for binding each of the affinity reagents that is most compatible with the plurality of binding outcomes for the extant proteins.

The present disclosure provides a detection system. The detection system can include (a) a detector configured to acquire signals from a plurality of binding reactions occurring between a plurality of different affinity reagents and a plurality of extant proteins in a sample; (b) a database including information characterizing or identifying a plurality of candidate proteins; (c) a computer processor configured to: (i) communicate with the database, (ii) process the signals to produce a plurality of binding profiles, wherein each of the binding profiles includes a plurality of binding outcomes for binding of an extant protein of (a) to the plurality of different affinity reagents, wherein individual binding outcomes of the plurality of binding outcomes include a measure of binding between an extant protein of (a) and a different affinity reagent of the plurality of different affinity reagents, each of the binding profiles including positive binding outcomes and negative binding outcomes, (iii) process the binding profiles to determine a probability for each of the affinity reagents binding to each of the candidate proteins in the database according to a binding model for each of the affinity reagents; and (iv) outputting an identification of selected candidate proteins, the selected candidate proteins being candidate proteins in the database having a probability for binding each of the affinity reagents that is most compatible with the plurality of binding outcomes for the extant proteins.

A method for identifying an extant protein can be carried out in a detection system. The method can include (a) acquiring signals from a plurality of binding reactions carried out in a detection system, wherein the binding reactions include contacting a plurality of different affinity reagents with a plurality of extant proteins in a sample; (b) processing the signals in the detection system to produce a plurality of binding profiles, wherein each of the binding profiles includes a plurality of binding outcomes for binding of an extant protein of step (a) to the plurality of different affinity reagents, wherein individual binding outcomes of the plurality of binding outcomes include a measure of binding between an extant protein of step (a) and a different affinity reagent of the plurality of different affinity reagents, each of the binding profiles including positive binding outcomes and negative binding outcomes; (c) providing as inputs to the detection system a database including information characterizing or identifying a plurality of candidate proteins; (d) providing as inputs to the detection system a binding model for each of the different affinity reagents; (e) processing the plurality of binding profiles in the detection system to determine a probability for each of the affinity reagents binding to each of the candidate proteins in the database according to the binding model; and (f) outputting from the detection system an identification of selected candidate proteins, the selected candidate proteins being candidate proteins in the database having a probability for binding each of the affinity reagents that is most compatible with the plurality of binding outcomes for the extant proteins.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications, patents, or patent applications incorporated by reference contradict the disclosure contained in the specification, the specification is intended to supersede and/or take precedence over any such contradictory material.

### BRIEF DESCRIPTION OF THE DRAWINGS

FIG. 1A shows a workflow from sample preparation to data analysis for a method of identifying proteins.

FIG. 1B shows a depiction of protein decoding resulting in identification of the protein at location A1 as EGFR.

FIG. 1C shows repeated sequential affinity reagent measurements on EGFR showing five unique binding patterns and one off-target binding event.

FIG. 1D shows number of affinity reagents sufficient for 90% human proteome coverage with variation in length of epitope (dimer, trimer, tetramer) and number of epitopes bound by each multi-affinity reagent (asterisk indicates a value>2,000).

FIG. 1E shows proteome coverage achieved as affinity reagent cycles are measured using either affinity reagents targeting trimer epitopes optimized for the human proteome or one of 20 random sets of trimer targets.

FIG. 1F shows proteome coverage for human, mouse, yeast, and E. coli proteomes measured with an affinity reagent set optimized for human proteome coverage.

FIG. 2A shows coverage of the human proteome for affinity reagents of varying binding affinity.

FIG. 2B shows coverage of the human proteome for affinity reagents of varying binding affinity with non-specific binding to an array surface. Circle area is proportional to proteome coverage (also labeled on circle).

FIG. 2C shows impact of mischaracterization of affinity reagent binding on proteome coverage for varying fraction of unknown high-affinity epitope targets. All error bars are standard deviation across five replicates.

FIG. 2D shows impact of mischaracterization of affinity reagent binding on proteome coverage for varying fraction of false high-affinity epitope targets identified. All error bars are standard deviation across five replicates.

FIG. 2E shows impact of mischaracterization of affinity reagent binding on proteome coverage for systematic measurement error in binding probability. All error bars are standard deviation across five replicates.

FIG. 2F shows impact of mischaracterization of affinity reagent binding on proteome coverage for random measurement error in binding probability. All error bars are standard deviation across five replicates.

FIG. 3A shows dynamic range of protein quantification for blood plasma with varying protein array size. Data are plotted in order of decreasing protein abundance from top to bottom. Dynamic range is the protein abundance divided by the most abundant protein in sample. The outer width of the contours indicates the percentage of proteins at that abundance deposited on the protein array (one or more copies). The inner width of the contours indicates the percentage of proteins at that abundance detected by the decoding method. Percentages are computed over a rolling window of 51 proteins. Horizontal gray bars indicate 100%.

FIG. 3B shows dynamic range of protein quantification for HeLa cells with varying protein array size. Data are presented as set forth above for FIG. 3A.

FIG. 3C shows reproducibility of quantification (coefficient of variation computed across five replicates) compared to protein abundance for plasma as contour plots (density iso-proportional contours) with marginal histograms.

FIG. 3D shows reproducibility of quantification (coefficient of variation computed across five replicates) compared to protein abundance for HeLa cells as contour plots (density iso-proportional contours) with marginal histograms.

FIG. 3E shows concordance of quantity of proteins (number of copies identified) measured by the decoding method with true count of protein on array for a single experimental replicate of plasma.

FIG. 3F shows concordance of quantity of proteins (number of copies identified) measured by the decoding method with true count of protein on array for a single experimental replicate of HeLa cells.

FIG. 4A shows impact of mischaracterization of affinity reagent binding on proteome coverage for varying fraction of unknown high-affinity (primary) epitope targets and low-to-medium affinity (secondary) epitope targets. All coverage measurements are the average over 5 replicates.

FIG. 4B shows varying fraction of false high-affinity (primary) and low-to-medium affinity (secondary) epitope targets identified. All coverage measurements are the average over 5 replicates.

FIG. 4C shows systematic measurement error in binding probability with varying fraction of the 300 total affinity reagents impacted by the corruption. All coverage measurements are the average over 5 replicates.

FIG. 4D shows random measurement error in binding probability with varying fraction of the 300 total affinity reagents impacted by the corruption. All coverage measurements are the average over 5 replicates.

FIG. 5A shows distribution of protein abundance among proteins in a sample, deposited on a protein array, and quantified by the decoding method in plasma measured on an array having 10¹⁰ protein-occupied addresses. Histogram counts for each group are averaged over five simulated replicate experiments. The displayed non-specific quant rate is the maximum percentage of proteins observed in any replicate with poor quantification (>10% signal arising from false identifications). The percent of proteins in the sample quantified is shown as a gray line. Mean proteome coverage is the percent of proteomes present in a sample detected by the decoding method (averaged across the five replicates). Error bars indicate standard deviation.

FIG. 5B shows distribution of protein abundance among proteins in a sample, deposited on a protein array, and quantified by the decoding method in depleted plasma measured on an array having 10¹⁰ protein-occupied addresses. Data was processed and presented for FIG. 5A.

FIG. 5C shows distribution of protein abundance among proteins in a sample, deposited on a protein array, and quantified by the decoding method in a HeLa cell line measured on an array having 10¹⁰ protein-occupied addresses. Data was processed and presented for FIG. 5A.

FIG. 5D shows distribution of protein abundance among proteins in a sample, deposited on a protein array, and quantified by the decoding method in plasma measured on an array having 10⁸ protein-occupied addresses. Data was processed and presented for FIG. 5A.

FIG. 5E shows distribution of protein abundance among proteins in a sample, deposited on a protein array, and quantified by the decoding method in depleted plasma measured on an array having 10⁸ protein-occupied addresses. Data was processed and presented for FIG. 5A.

FIG. 5F shows distribution of protein abundance among proteins in a sample, deposited on a protein array, and quantified by the decoding method in a HeLa cell line measured on an array having 10⁸ protein-occupied addresses. Data was processed and presented for FIG. 5A.

FIG. 6A shows sensitivity and specificity of the decoding method for non-depleted plasma. The probability threshold for protein identification was varied: log(threshold)=0, −1e-20, −1e-16, −1e-14, −1e-12, −1e-11, −1e-10, −1e-9, −1e-8, −1e-7, −1e-6, −1e-5, −1e-4, −1e-3, −1e-2, −0.1, −0.2, and −0.3. A low threshold resulted in higher sensitivity (proteins quantified) but also a higher rate of non-specific quantification (signals where 10% or more of identifications are false). A point is plotted indicating these metrics for each threshold assessed for each of 5 replicate samples (shown as varying shapes). Simulations were performed with datasets comprising 10¹⁰ protein-occupied addresses and 10⁸ protein-occupied addresses.

FIG. 6B shows sensitivity and specificity of the decoding method for depleted plasma. Data was processed and presented for FIG. 6A.

FIG. 6C shows sensitivity and specificity of the decoding method for a HeLa cell line. Data was processed and presented for FIG. 6A.

FIG. 7A shows dynamic range in abundance of proteins deposited on arrays of varying size for non-depleted plasma. Data are plotted in order of decreasing protein abundance from top to bottom. Dynamic range is the ratio of protein abundance to that of the most abundant in sample. Outer width of contours indicates percentage of proteins at that abundance deposited on array (1 or more copy) with the bar at the top of each contour corresponding to 100%. Percentages are computed over a rolling window of 51 proteins.

FIG. 7B shows dynamic range in abundance of proteins deposited on arrays of varying size for depleted plasma. Data was processed and presented for FIG. 7A.

FIG. 7C shows dynamic range in abundance of proteins deposited on arrays of varying size for HeLa cells. Data was processed and presented for FIG. 7A.

FIG. 8A shows dynamic range of protein quantification for a depleted blood sample evaluated using the decoding method. Protein abundance data are plotted in order of decreasing abundance from top to bottom. Dynamic range is the ratio of protein abundance to that of most abundant in sample. The outer width of the contours indicates the percentage of proteins at that abundance deposited on the array (one or more copies). The inner width of the contours indicates the percentage of proteins at that abundance detected by the decoding method. Percentages are computed over a rolling window of 51 proteins. Horizontal bars indicate 100%.

FIG. 8B shows reproducibility of quantification (CV % among five replicates) compared to protein abundance using a contour plot (density iso-proportional contours) with marginal histograms for a depleted blood sample evaluated using the decoding method.

FIG. 8C shows concordance of quantity of proteins (number of copies detected) with true count of protein on array for a single replicate of a depleted blood sample evaluated using the decoding method.

FIG. 8D shows distribution of fold-change error, which is the count of protein copies detected by the decoding method divided by copies of the depleted plasma proteins deposited on the array. Copies detected and copies deposited are averaged across five replicates measured.

FIG. 9A shows reproducibility and accuracy of quantification demonstrated for non-depleted plasma samples assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9B shows the concordance of quantity of proteins (number of copies identified) measured by the decoding method with true count of protein on array shown for a single replicate of non-depleted plasma. Copies detected and copies deposited are averaged across five replicates measured.

FIG. 9C shows the distribution of fold-change error, which is the count of protein copies identified by the decoding method divided by copies of the protein deposited on the array for the non-depleted plasma. Copies detected and copies deposited are averaged across five replicates measured.

FIG. 9D shows reproducibility and accuracy of quantification demonstrated for depleted plasma assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9E shows the concordance of quantity of proteins (number of copies identified) measured by the decoding method with true count of protein on array shown for a single replicate of depleted plasma. Copies detected and copies deposited are averaged across five replicates measured.

FIG. 9F shows the distribution of fold-change error, which is the count of protein copies identified by the decoding method divided by copies of the protein deposited on the array for the depleted plasma. Copies detected and copies deposited are averaged across five replicates measured.

FIG. 9G shows reproducibility and accuracy of quantification demonstrated for HeLa cells assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9H shows the concordance of quantity of proteins (number of copies identified) measured by the decoding method with true count of protein on array shown for a single replicate of HeLa cells. Copies detected and copies deposited are averaged across five replicates measured.

FIG. 9I shows the distribution of fold-change error, which is the count of protein copies identified by the decoding method divided by copies of the protein deposited on the array for HeLa cells. Copies detected and copies deposited are averaged across five replicates measured.

FIG. 10A shows the reproducibility of protein deposition and protein quantification across five replicates for non-depleted plasma measured on arrays with 10¹⁰ protein-occupied addresses. Protein quantity deposited is the total count of a protein that was successfully deposited on the array. Protein quantity measured is the number of times the protein was identified by the decoding method. The CV (%) of each of these quantities across the five replicates is computed for each unique protein detected in the sample and plotted using a contour plot to demonstrate the concordance of variation in protein counts deposited with variation in protein counts measured.

FIG. 10B shows the reproducibility of protein deposition and protein quantification across five replicates for HeLa cells measured on arrays with 10¹⁰ protein-occupied addresses. Data was processed and presented as set forth for FIG. 10A.

FIG. 11 shows fold-change measurement error distribution for proteins detected in plasma samples measured on 10¹⁰ protein-occupied addresses. Fold change error is the count of protein copies detected by the decoding method divided by copies of protein deposited on the array. Copies detected and copies deposited are averaged across five replicates measured.

FIG. 12 shows a computer system that is programmed or otherwise configured to implement a method set forth herein.

FIG. 13 shows predicted non-binding probabilities by sequence length for different semi-censored decode approaches.

FIG. 14 shows non-binding probability predictions for sequences of arbitrary length using different semi-censored decode approaches.

### DETAILED DESCRIPTION

A protein can be detected using one or more affinity reagents having known or measurable binding affinity for the protein. For example, an affinity reagent can bind a protein to form a complex and a signal produced by the complex can be detected. A protein that is detected by binding to a known affinity reagent can be identified based on the known or predicted binding characteristics of the affinity reagent. For example, an affinity reagent that is known to selectively bind a candidate protein suspected of being in a sample, without substantially binding to other proteins in the sample, can be used to identify the candidate protein in the sample merely by observing the binding event. This one-to-one correlation of affinity reagent to candidate protein can be used for identification of one or more proteins. However, as the protein complexity (i.e. the number and variety of different proteins) in a sample increases, the time and resources to produce a commensurate variety of affinity reagents having one-to-one specificity for the proteins approaches limits of practicality.

The present disclosure provides methods, systems and compositions that can be advantageously employed to overcome these constraints. In particular configurations, the number of different proteins identified can exceed the number of affinity reagents used. For example, the number of proteins identified can be at least 5×, 10×, 25×, 50×, 100× or more than the number of affinity reagents used. As set forth in further detail herein, one or more extant proteins can be identified by (1) performing binding reactions using promiscuous affinity reagents that bind to multiple different candidate proteins suspected of being present in a given sample, (2) subjecting one or more extant proteins to a set of the promiscuous affinity reagents that, taken as a whole, produce an empirical binding profile for each extant protein, and (3) performing a decoding method that evaluates the empirical binding profile according to a binding model for binding of the promiscuous affinity reagents to a plurality of candidate proteins, thereby identifying individual extant proteins based on compatibility with a respective candidate protein.

Promiscuity of an affinity reagent is a characteristic that can be understood relative to a given population of proteins. Promiscuity can arise due to the affinity reagent recognizing an epitope that is present in a plurality of different proteins that are known or suspected of being in a sample, such as a human proteome sample. For example, a promiscuous affinity reagent may recognize epitopes having relatively short amino acid lengths such as dimers, trimers, tetramers, pentamers or hexamers, wherein the epitopes are expected to occur in a substantial number of different proteins in a proteome of a human or other species. Alternatively or additionally, a promiscuous affinity reagent can recognize different epitopes (i.e. epitopes having a variety of different structures), the different epitopes being present in a plurality of different proteins in a proteome sample. For example, a promiscuous affinity reagent can have a high probability of binding to a primary epitope target and lesser probability for binding to one or more secondary epitope targets, the secondary epitope targets having a different sequence of amino acids when compared to the primary epitope target. Optionally, the secondary epitope targets can be biosimilar to the primary epitope target, for example, in accordance with a BLOSUM62 scoring matrix.

Although performing a single binding reaction between a promiscuous affinity reagent and a complex protein sample, such as a human proteome sample, may yield ambiguous results regarding the identity of the different proteins to which it binds, the ambiguity can be resolved when the results are evaluated in a decoding method set forth herein. A plurality of binding outcomes obtained from measuring binding of a plurality of affinity reagents with one or more extant proteins can be input into a decoding method of the present disclosure to identify the most likely identity of that protein among a set of candidate proteins. The plurality of binding outcomes can be input into a decoding method along with information characterizing or identifying a plurality of candidate proteins (e.g. amino acid sequences of candidate proteins), and a binding model. The probability of each affinity reagent binding to every possible candidate protein can be evaluated using the binding model and the decoding method can output the identity of individual extant proteins. For example, the decoding algorithm can output the most likely identity for an individual extant protein as the candidate protein that is most compatible with the observed binding outcomes for the extant protein according to the binding model.

A binding model of the present disclosure can be configured on an assumption that the characteristics for affinity reagents binding to extant proteins in a sample, even if unknown, can be treated as quantifiable random variables, and that uncertainty about the binding characteristics can be described by probability distributions. Parameters for a plurality of affinity reagents can be determined, for example, based on apriori knowledge about the affinity reagents (e.g. expected binding affinity for particular epitopes) and/or based on preliminary reactions performed using the affinity reagents (e.g. measurement of binding between the affinity reagents and one or more epitopes). The parameters of the affinity reagents can be treated as ‘priors’ that are input into a decoding algorithm of the present disclosure. The parameters of the affinity reagents when combined with empirically determined binding outcomes and evaluated using a decoding method of the present disclosure can output a ‘posterior,’ the calculation of which involves computation of a distribution of likelihoods for the identity of each extant protein used for the empirical determination. The posteriors that are output by the decoding method can be used to update the priors that will be used as inputs to subsequent evaluations using the decoding method. Accordingly, the influence of unknowns and artifacts in early evaluation of affinity reagents can be diminished as further empirical measurements are made and the results evaluated by the decoding method. This updating cycle can provide the benefit of facilitating iterative improvement to the decoding method, thereby improving the accuracy of identifying or characterizing extant proteins.

An advantage of the decoding method set forth herein is that it takes into account characteristics of binding reactions that may otherwise adversely affect the accuracy with which proteins can be identified. For example, binding reactions carried out at single-molecule scale (e.g. detecting binding of affinity reagents to proteins that are individually resolved on a protein array) produce stochastic results. Moreover, non-specific binding of affinity reagents, for example, to the surface of an array to which proteins under observation are attached, can also produce errant results. Another example is bias or skew that can arise due to different lengths of proteins that are analyzed in a decoding method set forth herein. A decoding method can be configured to account for stochasticity, non-specific binding, differences in protein length, or other factors for improved accuracy when identifying or characterizing proteins. For example, stochasticity can be accounted for by estimating protein likelihood using the decoding method. Similarly, differences in protein length can be accounted for by computing a normalization factor that depends jointly on candidate protein length and number of observed positive binding outcomes.

For ease of explanation, the compositions, systems and methods of the present disclosure are often exemplified herein in the context of characterizing proteins using binding measurements. The examples set forth herein can be readily extended to characterizing other analytes (e.g. as an alternative or addition to proteins), or to the performance of other reactions (e.g. as an alternative or addition to binding reactions).

The present disclosure provides compositions, systems and methods that can be useful in various configurations for characterizing analytes, such as proteins, nucleic acids, cells or moieties thereof, by obtaining multiple separate and non-identical measurements of the analytes. In particular configurations, the individual measurements may not, by themselves, be sufficiently accurate or specific to make the characterization, but an aggregation of the multiple non-identical measurements can allow the characterization to be made with a high degree of accuracy, specificity and confidence. In some cases, an aggregation of the multiple measurements using the same affinity reagent (e.g. repeating a binding reaction in triplicate) can allow characterization to be made with a high degree of accuracy, specificity and confidence. Optionally, a plurality of promiscuous reagents can be reacted with a given analyte and the reaction outcome observed for each of the promiscuous reagents can be detected. Promiscuous reagents can demonstrate both low specificity, with regard to the variety of different analytes recognized, and high reactivity for some or all of those analytes. Taking a binding reaction as an example, promiscuous affinity reagents can demonstrate both low specificity, with regard to the variety of different analytes recognized, and high affinity for some or all of those analytes. For any of a variety of reactions, including but not limited to binding reactions, a first reaction carried out using a first promiscuous reagent may perceive a first subset of analytes in a sample without distinguishing one analyte in the subset from another analyte in the sample. A second reaction carried out using a second promiscuous reagent may perceive a second subset of analytes in the sample, again, without distinguishing one analyte from another analyte in the second subset. However, a combination of measurements obtained from the first and second reactions can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the known or suspected diversity of different analytes for a given sample.

A composition, system or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full-length or fragmented), maturation state (e.g. presence or absence of pre- or pro-sequence in a protein), location (e.g. in an analytical system such as an array, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKₐ for a protein) or common moiety (e.g. a short primary sequence motif or post-translational modification for a protein). An analyte can be characterized with regard to a relatively specific characteristic such as a unique amino acid sequence (e.g. for the full-length of the protein or a motif), an RNA or DNA sequence that encodes a protein (e.g. for the full-length of the protein or a motif), or an enzymatic or other activity that identifies a protein. A characterization can be sufficiently specific to identify an analyte, for example, at a level that is considered adequate or unambiguous by those skilled in the art. An analyte can be identified with a probability or score surpassing a desired threshold for confident identification.

Methods, compositions and systems of the present disclosure can be advantageously deployed in situations where proteins yield different empirical binding profiles despite having identical primary structure and being subjected to the same set of affinity reagents. For example, the methods, compositions and systems are well suited for single-molecule detection and other formats that are prone to stochastic variability. Particular configurations of the compositions, systems and methods herein can overcome ambiguities and errors in observed binding outcomes to provide accurate identification and characterizations of proteins. The methods can be advantageously deployed for complex samples including proteomes or subfractions thereof.

Terms used herein will be understood to take on their ordinary meaning in the relevant art unless specified otherwise. Several terms used herein and their meanings are set forth below.

As used herein, the term “address” refers to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity reagent” or “binding reagent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the term “array” refers to a population of analytes (e.g. proteins) that are associated with unique identifiers such that the analytes can be distinguished from each other. A unique identifier can be, for example, a solid support (e.g. particle or bead), spatial address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “binding profile” refers to a plurality of binding outcomes for a protein or other analyte. The binding outcomes can be obtained from independent binding observations, for example, independent binding outcomes can be acquired using different affinity reagents, respectively. Alternatively, the outcomes can be statistical measures such as probabilities, likelihoods, measures of uncertainty or measures of variation. Optionally, the binding outcomes can be generated in silico, for example, being derived from a modification of an empirically obtained binding outcome. A binding profile can include empirical measurement outcomes, candidate measurement outcomes, putative measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes. The elements of the vector can be digital values (e.g. binary values representing positive and negative binding outcomes respectively) or analog values (e.g. probability values in a range from 0 to 1).

As used herein, the term “comprising” is intended to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “measurement outcome” refers to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome can include an empirical or putative measurement outcome for a candidate analyte (e.g. for a candidate protein) that is known or suspected of being present in a sample or assay. A measurement outcome can be represented in binary terms such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

As used herein, the term “promiscuous,” when used in reference to a reagent, means that the reagent is known or suspected to react with a variety of different analytes in a given sample. For example, an affinity reagent that is known or suspected to recognize a variety of different analytes (e.g. a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample.

As used herein, the term “protein” refers to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

The embodiments set forth below and recited in the claims can be understood in view of the above definitions.

The present disclosure provides a method of identifying an extant protein. The method can include steps of (a) providing inputs to a computer processor, the inputs including: (i) a binding profile, wherein the binding profile includes a plurality of binding outcomes for binding of the extant protein to a plurality of different affinity reagents, wherein individual binding outcomes of the plurality of binding outcomes include a measure of binding between the extant protein and a different affinity reagent of the plurality of different affinity reagents, the binding profile including positive binding outcomes and negative binding outcomes, (ii) a database including information characterizing or identifying a plurality of candidate proteins, and (iii) a binding model for each of the different affinity reagents; (b) determining a probability for each of the affinity reagents binding to each of the candidate proteins in the database according to the binding model; and (c) identifying the extant protein as a selected candidate protein, the selected candidate protein being a candidate protein in the database having a probability for binding each of the affinity reagents that is most compatible with the binding profile for the extant protein. Optionally, the inputs can further include (iv) a non-specific binding rate including a probability of a non-specific binding event occurring for one or more of the different affinity reagents.

Also provided is method of identifying an extant protein, which includes steps of: (a) contacting a plurality of different affinity reagents with a plurality of extant proteins in a sample; (b) acquiring binding data from step (a), wherein the binding data includes a plurality of binding profiles, wherein each of the binding profiles includes a plurality of binding outcomes for binding of an extant protein of step (a) to a plurality of different affinity reagents, wherein individual binding outcomes of the plurality of binding outcomes include a measure of binding between an extant protein of step (a) and a different affinity reagent of the plurality of different affinity reagents, each of the binding profiles including positive binding outcomes and negative binding outcomes; (c) providing a database including information characterizing or identifying a plurality of candidate proteins; (d) providing a binding model for each of the different affinity reagents; (e) determining a probability for each of the affinity reagents binding to each of the candidate proteins in the database according to the binding model; and (f) identifying the extant proteins as selected candidate proteins, the selected candidate proteins being candidate proteins in the database having a probability for binding each of the affinity reagents that is most compatible with the plurality of binding outcomes for the extant proteins.

The methods, compositions and systems of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins used herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or system set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or system set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or system set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or system set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or system set forth herein can have a complexity of at least 2, 5, 10, 100, 1×10¹, 1×10⁴, 1×10⁵, 1×10⁶, 1×10⁸, 1×10¹⁰, or more different primary protein structures. Alternatively or additionally, a plurality of proteins can have a complexity that is at most 1×10¹⁰, 1×10⁸, 1×10⁶, 1×10⁵, 5×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or system set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

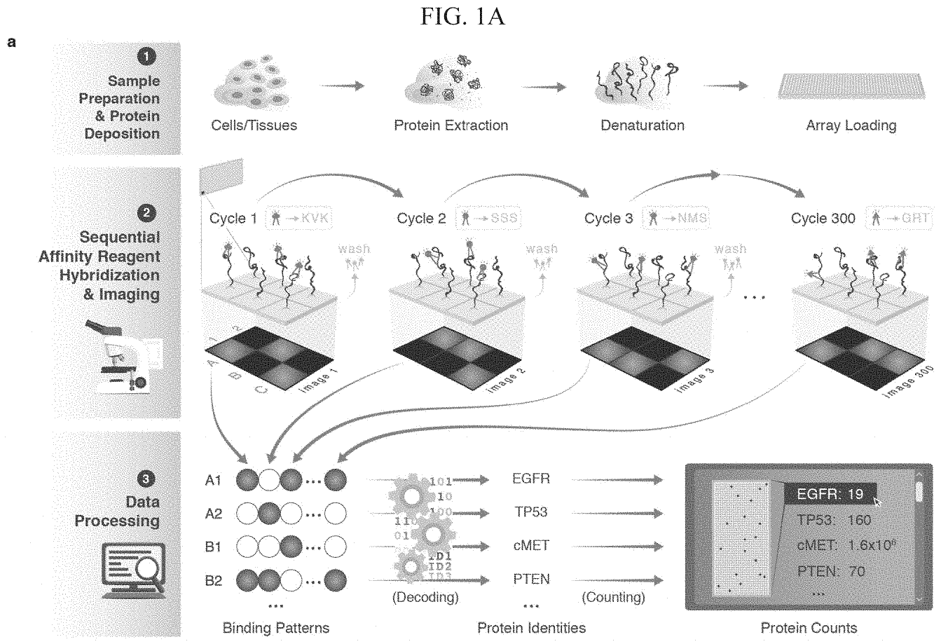

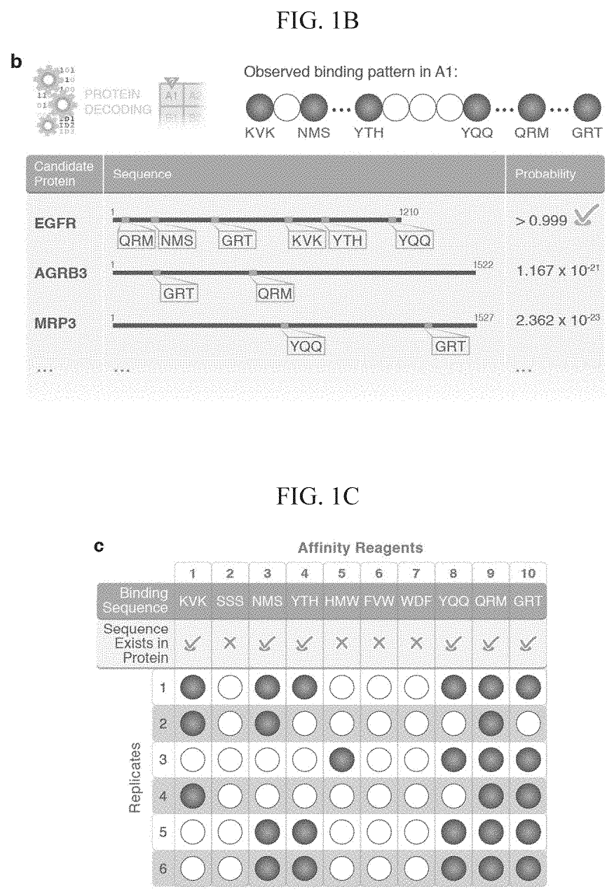

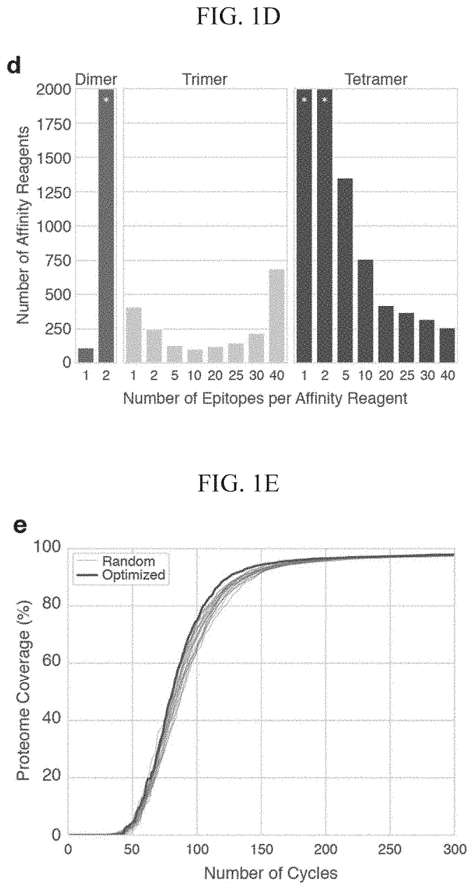

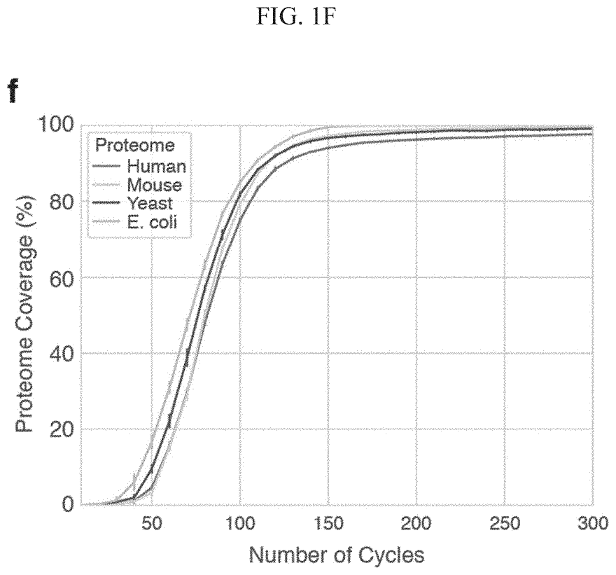

The present disclosure provides assays that are useful for detecting one or more analytes. An exemplary assay format is shown diagrammatically in FIG. 1A. Proteins can be extracted from a sample and attached to an array. Optionally, the unique identifiers of the array can be addresses. The array can be configured to have a plurality of addresses, wherein individual addresses are attached to individual proteins, respectively, from the sample. The proteins that are attached to the array can be in a denatured state or native state. Optionally, a structured nucleic acid particle (SNAP) can mediate attachment of each protein to its respective address. Other linkers or attachment chemistry that can be used additionally or alternatively to SNAPs include, but are not limited to, those set forth in US Pat. App. Pub. No. 2021/0101930 A1, WO 2021/087402 A1, or U.S. Pat. App. Ser. No. 63/159,500, each of which is incorporated herein by reference.

Typically, the identity of the protein at any given address is not known (as such, the proteins may be referred to as ‘unknown’ proteins). Methods set forth herein can be used to identify proteins at one or more addresses in the array. Accordingly, the methods can be used to locate extant proteins in an array. Continuing with the example diagrammed in FIG. 1A, a plurality of affinity reagents (e.g. antibodies, aptamers, or small proteins), tagged with fluorophores, can be contacted with the array, and fluorescence can be detected from individual addresses to determine binding outcomes. The affinity reagents can be delivered to the array and detected serially as shown, such that each cycle detects binding outcomes for an individual affinity reagent. In some configurations of the methods set forth herein, a plurality of different affinity reagents can be delivered in a cycle. The different affinity reagents that are delivered in a given cycle can be configured as a pool of indistinguishably labeled reagents (or they can lack labels), such that the different reagents are not distinguished in the detection step. Alternatively, two or more different affinity reagents that are delivered in a given cycle can be distinguishably labeled. As such the affinity reagents can be distinguishably detected when bound to proteins on the array. The use of fluorescent labels and fluorescent detection is exemplary. Other labels and other detectors can be used such as those set forth herein or known in the art.

Further examples of reagents and techniques that can be used to detect proteins in a method, system or composition of the present disclosure are set forth, for example, in U.S. Pat. No. 10,473,654 or US Pat. App. Pub. Nos. 2020/0318101 A1 or 2020/0286584 A1; or Egertson et al., BioRxiv (2021), DOI: 10.1101/2021.10.11.463967, each of which is incorporated herein by reference. Exemplary methods, systems and compositions are set forth in further detail below.

Some configurations of the compositions, systems or methods set forth herein, can distinguish different proteoforms, such as proteins having the same primary structure (i.e. the same sequence of amino acids) but differing with respect to the number, type, or location of post-translational modifications. Methods of the present disclosure can be configured to identify a number, type, or location for one or more post-translational modifications in one or more proteins of a sample. Exemplary post-translational modifications include, but are not limited to, a phosphoryl, glycosyl (e.g. N-acetylglucosamine or polysialic acid), ubiquitin, acyl (e.g. myristoyl or palmitoyl), isoprenyl, prenyl, farnesyl, geranylgeranyl, lipoyl, acetyl, alkyl (e.g. methyl or ethyl), flavin, heme, phosphopantetheinyl, C-terminal amidation, hydroxyl, nucleotidyl, adenylyl, uridylyl, proprionyl, S-glutathionyl, sulfate, succinyl, carbamyl, carbonyl, SUMOyl, or nitrosyl moiety.

Any of a variety of affinity reagents can be used in a composition, system or method set forth herein. An affinity reagent can be characterized, for example, prior to use in a method set forth herein, with respect to its binding properties. Exemplary binding properties that can be characterized include, but are not limited to, specificity, strength of binding; equilibrium binding constant (e.g. K_{A} or K_{D}); binding rate constant, such as association rate constant (kₒₙ) or dissociation rate constant (k_{off}); binding probability; or the like. Binding properties can be determined with regard to an epitope, a set of epitopes (e.g. a set of proteins having structural similarities), a protein, a set of proteins (e.g. a set of proteins having structural similarities), or a proteome.

An affinity reagent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label component may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

A method set forth herein can be carried out in a fluid phase or on a solid phase. For fluid phase configurations, a fluid containing one or more proteins can be mixed with another fluid containing one or more affinity reagents. For solid phase configurations one or more proteins or affinity reagents can be attached to a solid support. One or more components that will participate in a binding event can be contained in a fluid and the fluid can be delivered to a solid support, the solid support being attached to one or more other component that will participate in the binding event.

A method of the present disclosure can be carried out at single analyte resolution. A single analyte (e.g. a single protein) may be resolved from other analytes based on, for example, spatial or temporal separation from the other analytes. An alternative to single-analyte resolution is ensemble-resolution or bulk-resolution. Bulk-resolution configurations acquire a composite signal from a plurality of different analytes or affinity reagents in a vessel or on a surface. For example, a composite signal can be acquired from a population of different protein-affinity reagent complexes in a well or cuvette, or on a solid support surface, such that individual complexes are not resolved from each other. Ensemble-resolution configurations acquire a composite signal from a first collection of proteins or affinity reagents in a sample, such that the composite signal is distinguishable from signals generated by a second collection of proteins or affinity reagents in the sample. For example, the ensembles can be located at different addresses in an array. Accordingly, the composite signal obtained from each address will be an average of signals from the ensemble yet signals from different addresses can be distinguished from each other.

A composition, system or method set forth herein can be configured to contact one or more proteins (e.g. an array of different proteins) with a plurality of different affinity reagents. For example, a plurality of affinity reagents (whether configured separately or as a pool) may comprise at least 2, 5, 10, 25, 50, 100, 250, 500, 1000 or more types of affinity reagents, each type of affinity reagent differing from the other types with respect to the epitope(s) recognized. Alternatively or additionally, a plurality of affinity reagents may comprise at most 1000, 500, 250, 100, 50, 25, 10, 5, or 2 types of affinity reagents, each type of affinity reagent differing from the other types with respect to the epitope(s) recognized. Different types of affinity reagents in a pool can be uniquely labeled such that the different types can be distinguished from each other. In some configurations, at least two, and up to all, of the different types of affinity reagents in a pool may be indistinguishably labeled. Alternatively or additionally to the use of unique labels, different types of affinity reagents can be delivered and detected serially when evaluating one or more proteins (e.g. in an array).

A method of the present disclosure can be performed for a single analyte (e.g. a single protein gene product) or in a multiplex format. In multiplexed formats, in which the analytes are proteins, different proteins that are to be detected can be attached to different unique identifiers (e.g. addresses in an array), and the proteins can be manipulated and detected in parallel. For example, a fluid containing one or more different affinity reagents can be delivered to an array such that the proteins of the array are in simultaneous contact with the affinity reagent(s). Moreover, a plurality of addresses can be observed in parallel allowing for rapid detection of binding events. A plurality of different proteins can have a complexity of at least 5, 10, 100, 1×10¹, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, presence of unexpected proteins in a source for the sample, or loss of some proteins prior to analysis.

A particularly useful multiplex format uses an array of proteins and/or affinity reagents. A protein can be attached to a unique identifier (e.g. address of an array) using any of a variety of means. The attachment can be covalent or non-covalent. Exemplary covalent attachments include chemical linkers such as those achieved using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. at an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in US Pat. App. Pub. No. 2021/0101930 A1, WO 2021/087402 A1, or U.S. Pat. App. Ser. No. 63/159,500, each of which is incorporated herein by reference.

A method of the present disclosure can include a step of assaying binding between a protein and affinity reagent to determine a measurement outcome. For example, the measurement outcome for contacting an affinity reagent with an analyte can be observed as a binding outcome. The binding outcome can be positive or negative. For example, observation of binding is a positive binding outcome and observation of non-binding is a negative binding outcome. A binding outcome can be a null binding outcome, for example, when a positive binding outcome cannot be distinguished from a negative binding outcome.

Binding can be detected using any of a variety of techniques that are appropriate to the reaction components used. For example, binding can be detected by acquiring a signal from a label attached to an affinity reagent when the affinity reagents is bound to an observed protein, acquiring a signal from a label attached to protein when the protein is bound to an observed affinity reagent, or signal(s) from labels attached to an affinity reagent and protein when bound to each other. In some configurations a protein-affinity reagent complex need not be directly detected, for example, in formats where a nucleic acid tag or other moiety is created or modified as a result of binding between the protein and affinity reagent. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or US Pat. App. Ser. Nos. 63/112,607 or 63/132,170, each of which is incorporated herein by reference.

The present disclosure provides a decoding method, for example, in the form of a decoding algorithm, that can be used to evaluate the results of a binding reaction. The results can be used to identify or otherwise characterize proteins. In some configurations, distinct and reproducible binding profiles may be observed for some or even a substantial majority of proteins that are to be identified in a sample. However, in many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed individually. The present disclosure provides decoding methods that provide accurate protein identification despite ambiguities and imperfections that can arise in single-molecule formats or other contexts.

In some configurations, methods for identifying or characterizing one or more extant proteins in a sample utilize a decoding method that analyzes an empirical binding profile acquired for a plurality of binding reactions carried out between each extant protein in the sample and a plurality of affinity reagents, and then the empirical binding profile is evaluated with respect to the binding behavior of the affinity reagents to a plurality of candidate proteins. The plurality of candidate proteins can include proteins that are known or suspected of being present in the sample. Thus, the plurality of candidate proteins can include a plurality of native amino acid sequences. The decoding algorithm can output the identity of the extant protein as the candidate protein that has binding characteristics most compatible with the empirical binding profile. This compatibility can be determined based on a binding model that represents the affinity of each of the candidate proteins for each of the affinity reagents that were used to produce the empirical binding profile. A strong candidate protein can be identified as one for which the modeled binding outcomes are more consistent with the empirical binding profile as compared to the other candidate proteins evaluated.

A decoding method of the present disclosure can be configured to evaluate positive binding outcomes. In a censored decode configuration, the decoding method can evaluate positive binding outcomes without evaluating negative binding outcomes. In an uncensored decode configuration, a strong candidate protein can be identified as one for which a combination of positive binding outcomes and negative binding outcomes is more consistent with the empirical binding profile as compared to the other candidate proteins evaluated. A candidate protein can be identified as weak or even incorrect based on having many instances where positive binding outcomes and/or negative binding outcomes are inconsistent with the empirical binding profile being evaluated. The strongest candidate protein can be deemed the most likely identity for the extant protein and confidence in this identification can be computed as a relative measure of the compatibility of the most likely protein compared to all of the other candidate proteins.

A computer processor can be configured to execute a decoding method that outputs identities for one or more extant proteins based on various inputs. A particularly useful input is empirical binding data for binding of an extant protein to a plurality of different affinity reagents. The binding data can be in the form of an empirical binding profile that includes a plurality of binding outcomes. An empirical binding profile can include positive binding outcomes or negative binding outcomes. The same can be true for a candidate outcome profile. In some configurations a binding profile will include both positive binding outcomes and negative binding outcomes. For example, decoding can be carried out in an ‘uncensored’ configuration, wherein both positive and negative binding outcomes are considered. Alternatively, decoding can be carried out in a ‘censored’ configuration, wherein a subset of binding outcomes or a particular type of binding outcome is not considered. For example, a censored configuration can consider positive binding outcomes and omit negative binding outcomes. A censored approach can be useful, for example, in situations where there is an expectation that particular binding measurements or binding outcomes are prone to an unacceptable or undesirable level of errors or artifacts.

Uncensored decode can be configured to equally utilize both positive binding outcomes and negative binding outcomes when calculating the likelihood of a given extant protein having the identity of one or more candidate proteins. For example, the likelihood that each probe binds to each candidate protein can be known from empirical results and/or predicted from apriori determinations. The likelihood that each probe does not bind to each candidate protein can be determined simply as one minus the binding probability. The present disclosure provides a ‘semi-censored’ decoding configuration, wherein positive and negative binding outcomes are evaluated independent of each other. Semi-censored decode can be configured to treat negative binding outcomes as less informative than positive binding outcomes. Instead of treating a negative binding outcome as being informative about the amino acid sequence of an extant protein, the negative binding outcome can be treated as being informative about the length of the extant protein that was not bound. In some configurations of the methods set forth herein, semi-censored decode is premised on the presumption that shorter proteins will have fewer positive binding outcomes for a given set of affinity reagents compared to the number of positive binding outcomes for longer proteins.

For a semi-censored configuration, negative binding probabilities can be computed independent of computing positive binding probabilities. Semi-censored configurations provide the advantage of using a distinct method for updating protein likelihood from negative binding outcomes in comparison to the method used for positive binding outcomes. In a semi-censored configuration, positive binding outcomes can be weighted more heavily relative to negative binding outcomes. Alternatively, negative binding outcomes can be weighted more heavily relative to positive binding outcomes in a semi-censored configuration. The different weights can be applied to offset an expected or suspected bias in the binding reactions being evaluated, such as a high rate of off-target binding by one or more affinity reagents.

An empirical binding profile can be input to a decoding method set forth herein. For example, the empirical binding profile can be input to a computer processor that performs the decoding method. A series of empirical binding outcomes that constitute an empirical binding profile can be acquired using binding reactions such as those set forth herein or known in the art. Alternatively, a binding profile can be obtained from a simulation and used similarly to an empirical binding profile. Each empirical binding outcome in a binding profile can result from one binding reaction among a plurality of binding reactions carried out between an extant protein and a plurality of affinity reagents. An empirical binding profile can be decoded after all binding outcomes have been acquired for a given extant protein. Alternatively, for example, when binding outcomes are acquired serially, decoding can occur in real time such that evaluation of an empirical binding outcome from an earlier binding reaction in the series is initiated, and perhaps completed, prior to, or during, acquisition of an empirical binding outcome for a subsequent binding reaction in the series. A plurality of empirical binding outcomes need not necessarily be acquired serially, for example, instead being acquired such that some or all binding outcomes in an empirical binding profile are acquired from binding reactions that occur in parallel.

Another useful input to a decoding method is information for a plurality of candidate proteins. For example, information for a plurality of candidate proteins (e.g. a database of candidate protein information) can be input to a computer processor that performs the decoding method. A plurality of candidate proteins may include at least 10, 25, 50, 75, 100, 500, 1×10³, 1×10⁴, 1×10⁶, 1×10⁸ or more different candidate proteins. In some cases, a complete proteome or substantial fraction thereof can be included. For example, a database can include at least 10%, 25%, 50%, 75%, 90%, 95%, 99% or more of the proteins known, or suspected, to be present in a proteome set forth herein or known in the art. A database may include candidate proteins from more than one organism. For example, a database can include organisms from a given ecosystem such as a microbiome or environmental sample, organisms from a particular family, class or genera of species; or all known proteins from all known species.

Information that can be included in a database of candidate proteins includes, but is not limited to, primary structures (i.e. amino acid sequences), secondary structures, tertiary structures, quaternary structures, names, or other information pertaining to the candidate proteins. Optionally, a text-based format for representing amino acid sequences can be used as a database in a method or system set forth herein. Information provided in a FASTA format is particularly useful as a database. Optionally, information other than amino acid sequences can be included in a database. Particularly useful information that can be included in a database includes, for example, binding characteristics for binding of one or more affinity reagents to a protein. However, such information need not be included in the database and can instead be provided by a binding model. For example, the information can include a probability for each of a plurality of affinity reagents binding to each of a plurality of candidate proteins. In some configurations, such binding probabilities or other binding characteristics are derived empirically, for example, from binding experiments carried out between one or more known candidate proteins and one or more known affinity reagent(s). In some embodiments, binding probabilities or other binding characteristics are derived based on a priori information such as presence of a suspected epitope sequence in the primary structure (e.g. amino acid sequence) of a candidate protein. Any of a variety of publicly available databases can be used, such as those set forth in Example I, herein.

A database can include a probability or likelihood that a candidate protein would generate a positive binding outcome. Such information can be useful for several decoding configurations including, for example, censored, uncensored or semi-censored configurations. A database can further include a probability or likelihood that a candidate protein or pseudo protein would generate a negative binding outcome. Such information can be useful for an uncensored or semi-censored decoding configuration.

A binding model can be input to a decoding method set forth herein. For example, the binding model can be input to a computer processor that performs the decoding method. Optionally, a binding model can include a function for determining probability of a specific binding event occurring between a protein and each of a plurality of affinity reagents. In some configurations, a binding model can include a function for determining probability of a specific binding event occurring between a protein epitope and each of a plurality of affinity reagents. Epitopes evaluated by the model can have any of a variety of characteristics of interest. For example, the epitopes can have a defined length (e.g. the epitope length being less than or equal to 2, 3, 4, 5 or 6 amino acids in a protein primary sequence) or chemical composition (e.g. sequence of amino acids in a protein primary sequence). In some cases, the chemical composition can be relatively general with regard to chemical characteristics of amino acid side chains (or other moieties) such as charge, polarity, hydropathy, steric size, steric shape or the like. For example, the chemical composition of an epitope can be expressed in terms of biosimilarity to another epitope.

A decoding method set forth herein can include a function for calculating a probability of each affinity reagent binding to some or all possible candidate proteins among a plurality of candidate proteins in a given database. The function can consider positive binding outcomes. Optionally, the function can further consider negative binding outcomes, for example, when the function is used in an uncensored or semi-censored configuration. Optionally, binding probabilities can be configured as a matrix. As demonstrated in Example I, positive binding outcomes can be included in an M×N binding probability matrix B. In an uncensored configuration, the probability of a probe not binding to a protein can be expressed as: P(affinity probe not binding|protein)=1−P(affinity probe binding|protein). When using a binding probability matrix, a non-binding probability matrix U can be calculated as U=1−B. However, the uncensored approach may be adversely impacted by one or more non-binding events having an outsized impact on decoding. For example, an affinity reagent may not bind to a specific site for numerous difficult-to-predict reasons (e.g., protein structure, presence of unexpected post-translational modifications that hinder binding, etc.).

In some cases, decoding may be over-biased toward short proteins or long proteins. A normalization factor can be used to avoid over-biasing decoding results toward short or long proteins, thereby shifting likely identifications to overcome sequence length bias. In some cases, binding probabilities can be normalized for protein length by dividing the binding probabilities by a normalization constant. Another approach is to use a blinded uncensored approach in which uncensored decoding is adapted to be more resilient to missed binding events. This can be done by adjusting probabilities for negative binding outcomes. For example, probability of not binding a trimer of unknown identity can be computed for each affinity reagent:

θ=Σ₁⁸⁰⁰⁰p_{trimer_i}*(1−bp_{trimer_i})
with p_(trimer_i)=probability of the trimer appearing in the proteome (trimer_i frequency)/(total # trimers in proteome)
with bp_(trimer_i)=the binding probability of a probe to trimer_i,
b is not a constant in this instance
   The non-binding probability for a protein of length N can be set to:
θ^{N} (probability of non-binding to protein of length N of unknown trimer composition)

The above approach can be used to normalize proteins by length without considering specific trimer composition of each protein. The above approach can be readily adjusted for epitopes having other lengths. In another configuration, blinded uncensored decoding can be calculated as above for trimers, except a regression can be used to solve θ^{N}^{ⱼ} (1−P(probebindsproteinⱼ)) for θ using a plurality of different proteins as training points (NB “probe” means “affinity reagent” in this context). For example, 20,000 proteins can be used as training points in which case j=1 . . . 20,000. The above analysis can be modified for use with epitopes of sizes other than trimers, including for example, dimers, tetramers, pentamers etc.

A binomial approximation can be used for length normalization. The approximation can be carried out by counting the total number of possible specific binding events S and total number of possible non-specific binding events NS; computing the average binding probability among possible specific binding events: s̅; computing the average binding probability among possible non-specific binding events: n̅s̅; for a set of observed binding events, counting the number of observed specific (Oₛ) and observed non-specific (Oₙₛ) events (using same classification metric); and computing probability of observed binding event counts for candidate proteins as Binom(S, s̅).pmf(Oₛ)*Binom(NS, n̅s̅).pmf(Oₙₛ). In some cases, when decoding a protein address with N observed binding events, only the proteins with reasonable probability of generating observed binding event counts are considered. Optionally, binomial approximation can be included in a semi-censored decoding configuration, such as those set forth herein.

Length normalization can employ a Poisson binomial (e.g. exact or estimated Poisson binomial). Normalization can be carried out as follows. For a protein with binding probabilities p={p₁, p₁, p₁ . . . p₃₀₀} compute the probability of observing N binding events using the pmf of the Poission-binomial distribution parameterized by p; for each candidate protein, multiply likelihood of observed binding events by PoiBin(p).pmf(N). The Poisson binomial pmf can be calculated using an “exact” computation method or a refined-normal approximation (normal distribution+skew) (See Hong et al., Computational Statistics & Data Analysis 59:41-51 (2013), which is incorporated herein by reference)

Length normalization can also be performed via a semi-censored approach as set forth herein. A semi-censored configuration can allow the total number of non-binding events to be considered more than the specific identity of the observed non-binding events. Example I demonstrates a semi-censored configuration in which non-binding probabilities are adjusted to account for salient characteristics of candidate proteins such as length of the candidate proteins and relative frequency of every possible unique epitope of a particular amino acid length (e.g. dimer, trimer, tetramer etc.). A vector of average non-binding probabilities for affinity reagents can be calculated. For example, the probability of a given affinity reagent not binding to a trimer epitope, averaged over all 8000 trimers and weighted by the relative frequency of each trimer in the candidate protein database can be calculated.

Another approach that can be used to avoid over-biasing decoding results toward short or long proteins is to configure a semi-censored decoding method to predict the probability of negative binding outcomes based on the length of proteins suspected of being in a sample but agnostic to the amino acid sequences of the proteins. Optionally, the prediction can also be made independent of knowledge of the epitopes for the affinity reagents that are used to assay the sample. For example, the probability of negative binding outcomes can be predicted independent of the sequence length for the epitopes. As such, decoding can be based on an algorithm that is equally applicable to use of dimer, trimer, tetramer or other length epitopes. As set forth in further detail below, a set of pseudo proteins can be generated and the set can be used to predict negative binding probabilities.

A semi-censored decoding method can be configured to use a plurality of candidate proteins that includes amino acid sequences that are known or suspected of being present in a given sample. For example, a decoding method that is configured to evaluate proteins from a human can utilize a plurality of candidate proteins that include amino acid sequences that are native to humans. A semi-censored decoding method can be further configured to use a set of pseudo proteins that can optionally differ from the set of candidate proteins. A plurality of candidate proteins having native sequences can be useful for determining probabilities for positive binding outcomes between affinity reagents and candidate proteins. A plurality of pseudo proteins can be useful for determining probabilities for negative binding outcomes between affinity reagents and candidate proteins.

In some configurations, the set of pseudo proteins can include full-length amino acid sequences that are known or suspected to not be present in a given sample. For example, none of the full-length amino acid sequences in the set of pseudo proteins need be present in the set of candidate proteins and vice versa. Alternatively, a single full-length amino acid sequence or subset of amino acid sequences can be present both in a set of pseudo proteins and in a set of candidate proteins. In some configurations, partial amino acid sequences can be present both in a set of pseudo proteins and in a set of candidate proteins. The partial sequences that are present in both sets can contain at most 50, 40, 30, 20, 10, 9, 8, 7, 6, 5, 4 or 3 sequential amino acids. Alternatively or additionally, the partial sequences that are present in both sets can contain at least 3, 4, 5, 6, 7, 8, 9, 10, 20, 30, 40, or 50 sequential amino acids. In yet other configurations, the same amino acid sequences, whether full-length or partial, can be present both in a set of pseudo proteins and in a set of candidate proteins.

Turning to the example of a decoding method that is configured to evaluate proteins from a particular organism, a set of pseudo proteins can be utilized that includes amino acid sequences that are not native to the organism. For example, the set of pseudo proteins can include amino acid sequences that are native to one or more organisms other than the organism under evaluation. Optionally, a plurality of candidate proteins can lack full-length amino acid sequences that are non-native to a given sample (e.g. non-native to a particular organism) and a plurality of pseudo proteins can lack amino acid sequences that are native to the given sample (e.g. native to a particular organism).

When performing a semi-censored decoding method, the number of pseudo proteins can be substantially the same as the number candidate proteins. For example, the plurality of candidate proteins can include native sequences for proteins that are known or suspected of being in a given sample, and the plurality of pseudo proteins can include an amino acid sequence related to each of the native sequences in the plurality of candidate proteins. The pseudo amino acid sequences can be related to respective native sequences by virtue of each pseudo amino acid sequence having full-length that is the same as the full-length for a native amino acid sequence among the candidate proteins. However, each pseudo sequence can optionally differ from its related native sequence in terms of the amino acid content of the sequences.

In an alternative configuration, the number of pseudo proteins utilized in a semi-censored decoding method can be greater than the number candidate proteins utilized. For example, the plurality of candidate proteins can include native sequences for proteins that are known or suspected of being in a given sample, and the plurality of pseudo proteins can include multiple pseudo sequences related to each of the native sequences. Individual native sequences in a plurality of candidate proteins can each be related to at least 2, 3, 4, 5, 10, 25 or more pseudo sequences in a plurality of pseudo proteins. Again, the pseudo sequences can be related to respective native sequences in terms of the length for the two sequences. However, each pseudo sequence can differ from its related native sequence in terms of amino acid content.

A set of pseudo proteins can be generated using any of a variety of methods. For example, pseudo amino acid sequences can be selected at random. By way of more specific example, a pseudo sequence can be generated for an individual native sequence by scrambling the order of amino acids in the native sequence. Another option is to generate a pseudo sequence for an individual native sequence by randomly assigning one of the 20 native amino acids to each position along the length of the native sequence.

Optionally, a set of pseudo sequences can be generated in a way that biases or weights the pseudo amino acid sequences to reflect characteristics of a plurality of native amino acid sequences that are present in a proteome or other sample that is to be evaluated using a decoding method set forth herein. For example, a binning approach can be used in which all candidate proteins for a given sample (e.g. all proteins in a proteome) are aggregated into bins according to their amino acid sequence lengths. Within each bin the uncensored non-binding likelihood can be predicted for each protein and the median value can be used as the semi-censored non-binding likelihood for the entire bin. The proteins in the bin are thus representative of sequence biases in the sample.

Another approach that can be used is to create a set of pseudo sequences that are representative of sequence biases in a proteome (or other sample) of interest and to predict non-binding probabilities for the pseudo sequences. For example, a Markov model can be used. A Markov model is a statistical technique that can be used to model sequences such that the probability of a sequence element is based on a limited context preceding the element. A Markov model can be used to factorize the probability of observing an amino acid sequence in terms of context-dependent probabilities of amino acids in the sequence. A collection of pseudo sequences can be generated by a Markov chain Monte Carlo sampling of amino acid sequences in a plurality of native sequences as set forth in Example II below.

A Markov chain can be adjusted to suit a particular assay condition or sample. For example, transition probabilities can be modified to account for over-representation or under-representation of one or more proteins in a sample. This approach can be useful, for example, when a sample is experimentally enriched for one or more protein sequences. Thus, a protein sample can be fractionated, for example, via immunoprecipitation, chromatography or other known separation technique, and assay results for the fractionated sample can be decoded with a set of pseudo proteins derived from use of appropriately modified transition probabilities in a Markov chain algorithm. Similarly, modified transition probabilities can be used to account for changes in a proteome resulting from over-expression or under expression of one or more proteins as can occur from certain diseases (e.g. cancer) of from genetic engineering.

Another algorithm that can be used is generative adversarial network (GAN). For example, a GAN can generate a set of pseudo proteins from a set of candidate proteins such that the set of pseudo proteins has similar amino acid sequence characteristics as the set of candidate proteins. In some cases, a GAN can generate a set of pseudo proteins from a set of proteins other than the set of candidate proteins that will be used for a decoding method. For example, a GAN can generate a set of pseudo proteins based on a subset of amino acid sequences within the set of candidate proteins that will be used for decoding, based on a larger set of amino acid sequences that includes some or all sequences in the set of candidate proteins that will be used for decoding, or based on a set of amino acid sequences from an organism other than the organism for the candidate proteins that will be used for decoding. An expectation maximization algorithm can also be used to generate a set of pseudo proteins.

A plurality of pseudo proteins can have a total amino acid composition that is substantially equivalent to the amino acid composition of a plurality of candidate proteins. In another example, a plurality of pseudo proteins can have a total composition of amino acid k-mers (e.g. dimers, trimers, tetramers, pentamers etc.) that is substantially equivalent to the total composition of the amino acid k-mers in a plurality of candidate proteins. A plurality of pseudo proteins can have sequence bias that is substantially equivalent to sequence bias in a plurality of candidate proteins. For example, the dependencies of particular k-mers on their sequence context can be the same in the plurality of pseudo proteins as in the plurality of candidate proteins. In this example, sequence context can refer to the type of single amino acid that is upstream or downstream of the k-mer. In some cases, the sequence context can refer to a subsequence of two or more amino acids that occur upstream or downstream of the k-mer.

Accordingly, a method of identifying an extant protein can include steps of: (a) providing inputs to a computer processor, the inputs including: (i) a binding profile, wherein the binding profile includes a plurality of binding outcomes for binding of the extant protein to a plurality of different affinity reagents, wherein individual binding outcomes of the plurality of binding outcomes include a measure of binding between the extant protein and a different affinity reagent of the plurality of different affinity reagents, the binding profile including positive binding outcomes and negative binding outcomes, (ii) a database including information characterizing or identifying a plurality of candidate proteins, and (iii) a binding model for each of the different affinity reagents; (b) determining a probability for each of the affinity reagents binding to candidate proteins in the database according to the binding model, wherein the determining comprises computing probabilities for the positive binding outcomes and for the negative binding outcomes, and wherein the positive binding outcomes are weighted more heavily relative to the negative binding outcomes; and (c) identifying the extant protein as a selected candidate protein, the selected candidate protein being a candidate protein in the database having a probability for binding each of the affinity reagents that is most compatible with the binding profile for the extant protein. Optionally, step (b) can include (i) computing probability for a positive binding outcome occurring between each of the candidate proteins and each of the affinity reagents, and (ii) computing probability of a negative binding outcome occurring between each pseudo protein in a plurality pseudo proteins and each of the affinity reagents.

In an optional configuration of the above method, amino acid sequences in the plurality of pseudo proteins have full-lengths that are identical to the full-lengths for amino acid sequences in the plurality of candidate proteins. As a further option, the plurality of pseudo proteins can lack some or all full-length amino acid sequences that are present in the plurality of candidate proteins. Further optionally, amino acid sequences in the plurality of pseudo proteins can be generated by sampling of amino acid sequences in the plurality of candidate proteins using a Markov chain, generative adversarial network or length-based binning.

The plurality of candidate proteins used in a method set forth herein can include amino acid sequences that are native to a sample from which an extant protein of interest is derived whereas a plurality of pseudo proteins can include amino acid sequences that are not native to the sample. Optionally, individual pseudo proteins of the plurality of pseudo proteins can each have a full-length that is the same as the full-length of a candidate protein in the plurality of candidate proteins.

A decoding method set forth herein can include a function for determining probability of a non-specific binding event occurring between a protein and a plurality of affinity reagents. The model can account for the context of one or more epitopes in a given candidate protein. For example, a function for determining probability can be normalized with respect to the length of the given candidate protein. Alternatively or additionally, a binding model used in a method or system set forth herein can include a function for determining probability of a specific binding event occurring between a candidate protein and each of the affinity reagents. Again, the model can account for the context of one or more epitopes in a given candidate protein. For example, the function can be normalized with respect to the length of the given candidate protein.

In some configurations, a decoding method can include a function for determining probability of a binding event occurring between each of the affinity reagents and an epitope that is biosimilar to a specific epitope for the respective affinity reagent. In a biosimilar model, an affinity reagent can be considered as targeting a specific epitope to which it binds with particular probability. For example, the probability can be at least 0.01, 0.05, 0.1, 0.25 0.5, 0.75, 0.9, 0.99 or higher. Alternatively or additionally, the probability can be at most 0.99, 0.9, 0.75, 0.5, 0.25, 0.1, 0.05, 0.01 or lower. The affinity reagent can also be considered to bind one or more additional primary off targets with a probability in a range above. The number of additional primary targets can be at least 1, 3, 5, 7, 9, 15, 20 or more epitopes that are biosimilar to the targeted epitope. Alternatively or additionally, the number of additional primary targets can be at most, 20, 15, 9, 7, 5, 3 or 1 epitopes that are biosimilar to the targeted epitope. Biosimilar epitope targets can be selected by computing a pairwise similarity score of the target epitope to every other possible epitope of the same length and then selecting one or more of the other epitopes with a high similarity score. A similarity score can be computed by summing up similarity between the pair of residues at each sequence location, for example, using BLOSUM62 or other function for determining biosimilarity.

A parameterized binding model can be used in a decoding method of the present disclosure. For example, an affinity reagent can be modeled by assigning a binding probability to each unique target epitope recognized by the affinity reagent. Optionally, a non-specific binding rate can be assigned to individual affinity reagents. The non-specific binding rate can, for example, represent probability of a given affinity reagent binding to any epitope in a protein non-specifically. The probability of an affinity reagent binding to a given candidate protein can be computed by first computing the probability of a specific binding event happening. The model can consider the count of each epitope in a given protein sequence. The binding model parameters can include a vector of probabilities of a given affinity reagent binding to each recognized epitope. Furthermore, the model can include a function for computing the probability of a non-specific protein binding event happening. Optionally, the model can take into account the length of each candidate protein sequence, the length of an epitope recognized by the affinity reagent or both. The probability of the affinity reagent binding to the protein and generating a detectable signal can be represented as the probability of one or more specific or non-specific binding events occurring. Exemplary binding models are provided in Example I herein.

In some configurations of a system or method set forth herein, a non-specific binding rate can be provided as an input. The input can be in the form of one fixed non-specific binding rate for all affinity reagents, or a unique non-specific binding rate for each affinity reagent. Also, non-specific binding rate can be learned iteratively and/or adaptively in the same manner as other parameters in an affinity reagent binding model. The non-specific binding event can be binding of an affinity reagent to a substance other than a protein. The substance can be a solid support attached to an extant protein. For example, a non-specific binding event can occur at a region of an array where no protein of interest resides, such as a location at or near an address where a protein of interest resides. In some cases, a non-specific binding event can occur at an empty address, where a protein does not reside or at an interstitial region on the array that separates one address from another. Optionally, as exemplified in Example I herein, the input can be a surface non-specific binding rate describing the probability of a surface non-specific binding event happening in any given cycle in a series of binding reactions.

Execution of a decoding algorithm can include computing a probability matrix that includes the probabilities of a positive binding outcome for individual affinity reagents binding to each candidate protein used in a binding reaction. Optionally, the method can further include computing a probability matrix that includes the probabilities of a negative binding outcome for individual affinity reagents binding to each candidate protein used in a binding reaction. For example, adjusted non-binding probabilities can be computed as set forth in Example I or Example II, herein. In an alternative configuration of systems and methods set forth herein, the probabilities of a negative binding outcome can be calculated by subtracting the probabilities of a positive binding outcome from 1, the probabilities being represented by a value between 0 and 1. Positive and negative binding outcomes can be equally weighted. Alternatively, positive binding outcomes can be weighted more heavily relative to negative binding outcomes. In other cases, negative binding outcomes can be weighted more heavily relative to positive binding outcomes. The latter weighting can be particularly desirable to account for the numerous difficult-to-predict mechanisms by which an affinity reagent may bind to proteins non-specifically.

Decoding can be carried out by computing a vector of likelihoods for a plurality of candidate proteins. The candidate protein of highest likelihood can be selected. For example, the selected candidate protein can be the one having the most probabilities for binding the affinity reagents that are consistent with most of the binding outcomes obtained for a given extant protein. In another example, a candidate protein can be selected by multiplying the probabilities of the observed binding outcomes. Optionally, if there was a tie for top protein, one of the top proteins can be selected randomly or by another desired criteria. The probability of an identification being correct can be based on the likelihood of the top protein being correct divided by the sum of the likelihood of all other candidate proteins being correct. The protein identity can be output from the decoding system or method. Optionally, the probability of an identification being correct can be output. The probability can be calculated as the quotient of dividing the likelihood of a selected candidate protein by the sum of the likelihoods determined for all the other candidate proteins that were evaluated by the decoding algorithm.

Exemplary algorithms, and methods for characterizing proteins that can be used in combination with a method or system set forth herein include, for example, those set forth in US Pat App. Pub. No 2020/0286584 A1 or Egertson et al., BioRxiv (2021), DOI: 10.1101/2021.10.11.463967, each of which is incorporated herein by reference.

A decoding method can output information pertaining to the identity for one or more extant proteins. The information output for a given protein can be in the form of a determined identity for the protein or in the form of a probability or likelihood for one or more identity of the protein. For example, the most likely identity for an extant protein, the likelihood or probability of the extant protein having a particular identity, or both can be output by a decoding method. A decoding method can output a non-digital or non-binary score for the identity of a given extant protein or for the likelihood of the extant protein having a particular identity. For example, probability or likelihood scores can be output in the form of an analog value between 0 and 1, or percent value between 0% and 100%. In some configurations, a digital or binary score that indicates one of two discrete states can be output to indicate the identity of a protein or at least a subset of proteins (e.g. a family of proteins sharing a common structural motif) to which the protein belongs.

One or more steps of a method set forth herein can be carried out in a detection system. Accordingly, a detection system can be configured to execute one or more steps of a method set forth herein. For example, a detection system can be configured to execute one or more steps of a decoding method set forth herein. A decoding method set forth herein can be configured to improve the accuracy of the detection system. For example, the detection system can provide an initial identity or characterization for one or more extant proteins and a decoding method set forth herein can be used to output a subsequent identity or characterization that is more accurate or otherwise improved compared to the initial identity or characterization.

The present disclosure provides a detection system that include (a) a detector configured to acquire signals from a plurality of binding reactions occurring between a plurality of different affinity reagents and a plurality of extant proteins in a sample; (b) a database including information characterizing or identifying a plurality of candidate proteins; (c) a computer processor configured to: (i) communicate with the database, (ii) process the signals to produce a plurality of binding profiles, wherein each of the binding profiles includes a plurality of binding outcomes for binding of an extant protein of (a) to the plurality of different affinity reagents, wherein individual binding outcomes of the plurality of binding outcomes include a measure of binding between an extant protein of (a) and a different affinity reagent of the plurality of different affinity reagents, each of the binding profiles including positive binding outcomes and negative binding outcomes, (iii) process the binding profiles to determine a probability for each of the affinity reagents binding to each of the candidate proteins in the database according to a binding model for each of the affinity reagents; and (iv) outputting an identification of selected candidate proteins, the selected candidate proteins being candidate proteins in the database having a probability for binding each of the affinity reagents that is most compatible with the plurality of binding outcomes for the extant proteins.

A method for identifying an extant protein can be carried out in a detection system. The method can include (a) acquiring signals from a plurality of binding reactions carried out in a detection system, wherein the binding reactions include contacting a plurality of different affinity reagents with a plurality of extant proteins in a sample; (b) processing the signals in the detection system to produce a plurality of binding profiles, wherein each of the binding profiles includes a plurality of binding outcomes for binding of an extant protein of step (a) to the plurality of different affinity reagents, wherein individual binding outcomes of the plurality of binding outcomes include a measure of binding between an extant protein of step (a) and a different affinity reagent of the plurality of different affinity reagents, each of the binding profiles including positive binding outcomes and negative binding outcomes; (c) providing as inputs to the detection system a database including information characterizing or identifying a plurality of candidate proteins; (d) providing as inputs to the detection system a binding model for each of the different affinity reagents; (e) processing the plurality of binding profiles in the detection system to determine a probability for each of the affinity reagents binding to each of the candidate proteins in the database according to the binding model; and (f) outputting from the detection system an identification of selected candidate proteins, the selected candidate proteins being candidate proteins in the database having a probability for binding each of the affinity reagents that is most compatible with the plurality of binding outcomes for the extant proteins.

A detection system can include a detector, such as those known in the art for detecting a label or analyte set forth herein. A detector can be configured to collect signals (e.g. optical signals) from an array or other vessel containing extant proteins or other analytes. A camera such as a complementary metal-oxide-semiconductor (CMOS) or charge-coupled device (CCD) camera can be particularly useful, for example, to detect optical labels such as luminophores. The detection system can further include an excitation source configured to excite extant proteins, affinity reagents or other analytes, for example, in an array or other vessel. A detection system can include a scanning mechanism configured to effect relative movement between a detector and an array or other vessel containing extant proteins. Optionally, the scanning mechanism can be configured for time-delayed integration. Detectors that are capable of resolving proteins on an array surface including, for example, at single-molecule resolution can be particularly useful. Detectors used in DNA sequencing systems can be modified for use in a detection system or other apparatus set forth herein. Exemplary detectors are described, for example, in U.S. Pat. Nos. 7,057,026; 7,329,492; 7,211,414; 7,315,019 or 7,405,281, or US Pat. App. Pub. No. 2008/0108082 A1, each of which is incorporated herein by reference.

A detection system can further include fluidics apparatus configured to contact reaction components for a reaction or other step of a method set forth herein. In particular embodiments, reactions occur on arrays. Any of a variety of arrays can be present in the system, such as an array set forth herein. Proteins that are to be detected, for example those attached to an array, can be housed in any of a variety of reaction vessels. A particularly useful reaction vessel is a flow cell. A flow cell or other vessel can be present in a system in a permanent manner or in a removable manner, for example, being removable by hand or without the use of an auxiliary tool. A flow cell or other vessel can have a detection window through which a detector observes one or more proteins (e.g. an array of proteins) or other analytes on an array. For example, an optically transparent window can be used in conjunction with an optical detector such as a fluorimeter or luminescence detector.

A fluidic apparatus can include one or more reservoirs which are fluidically connected to an inlet of a flow cell or other vessel. The reservoirs can include reagents for use in a method set forth herein. The system can further include a pump, pressure supply or other fluid displacement apparatus for driving reagents from reservoirs to the vessel. The system can include a waste reservoir that is fluidically connected to an egress of a vessel to remove spent reagents. Taking as an example an embodiment where the vessel is a flow cell, reagents can be delivered to the flow cell through a flow cell ingress and then the reagents can flow through the flow cell and out the flow cell egress to a waste reservoir. Accordingly, the flow cell can be in fluidic communication with one or more reservoirs of the system. A fluidic system can include at least one manifold and/or at least one valve for directing reagents from reservoirs to a vessel where detection occurs. Exemplary fluidic apparatus that can be used in a system of the present disclosure include those configured for cyclic delivery of reagents, such as those deployed in nucleic acid sequencing reactions. Exemplary fluidic apparatus are set forth in US Pat. App. Pub. Nos. 2009/0026082 A1; 2009/0127589 A1; 2010/0111768 A1; 2010/0137143 A1; or 2010/0282617 A1; or U.S. Pat. Nos. 7,329,860; 8,951,781 or 9,193,996, each of which is incorporated herein by reference.

The present disclosure provides computer systems (e.g. computer control systems) that are programmed to implement methods, algorithms or functions set forth herein. Optionally, a computer system set forth herein can be a component of a detection system. A computer system can be programmed or otherwise configured to: (a) receive an input set forth herein such as a binding profile, a database comprising information characterizing or identifying a plurality of candidate proteins, a binding model and/or non-specific binding rates for affinity reagents, (b) determine probabilities for affinity reagents binding to candidate proteins, for example, based on a binding model, and (c) identify extant proteins as selected candidate proteins.

FIG. 12 shows an exemplary computer system 1001. The computer system 1001 can be an electronic device of a detection system, the electronic device being integral to the detection system or remotely located with respect to the detection system. For example, the electronic device can be a mobile electronic device. The computer system 1001 includes a computer processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. For example, one or more computer servers may enable cloud computing over the network 1030 (“the cloud”) to perform various aspects of analysis, calculation, and generation of the present disclosure, such as, for example, receiving information of empirical measurements of extant proteins in a sample; processing information of empirical measurements against a database comprising a plurality of protein sequences corresponding to candidate proteins, for example, using a binding model or function set forth herein; generating probabilities of a candidate protein generating empirical measurements, and/or generating probabilities that extant proteins are correctly identified in the sample. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome set, and/or generate probabilities that candidate proteins are correctly identified in the sample.

The present disclosure provides a non-transitory information-recording medium that has, encoded thereon, instructions for the execution of one or more steps of the methods set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Example I

### Single-Molecule Protein Identification Using Multi-Affinity Protein Affinity Reagents

This example describes a foundation for high-throughput single-molecule protein identification. The approach uses multi-affinity reagents that bind short, linear epitopes with low specificity and a decoding algorithm that accommodates stochasticity expected for single-molecule binding. In simulations, the approach achieved high proteome coverage in a wide range of organisms and was robust to potential experimental confounders. Simulating a human blood plasma proteome experiment, the approach supported a dynamic range of detection spanning at least eight orders of magnitude. The results indicated that, if executed experimentally, the approach could quantitatively decode over 90% of the human proteome in a single experiment, potentially revolutionizing proteomics research.

### Results and Discussion

As a preliminary matter, the present example sets forth methods that can be used to identify and distinguish proteins based on their primary structure (i.e. amino acid sequence). In this context, reference to proteins differing, whether implied or explicit, pertains to differences in their primary structure. Notwithstanding the foregoing, the methods exemplified herein can be useful, in some cases by adaptation that will be apparent to those skilled in the art, to identifying proteins based on differences such as presence, number, type or location of post translational modifications.

FIG. 1A shows an experimental setup for detecting a plurality of proteins at single molecule resolution. Proteins are extracted from a sample and each protein is conjugated in a denatured state to a structured nucleic acid particle (SNAP) followed by deposition of the protein-conjugated SNAP on a solid support having 10¹⁰ addresses. No more than one protein-conjugated SNAP binds per address, creating a hyper-dense single molecule array with each address having a protein that is optically resolvable from neighboring addresses. A series of affinity reagents (e.g. antibodies, aptamers, or small proteins), tagged with fluorophores, is contacted with the array. One affinity reagent is used per cycle of the series, presence or absence of binding is detected at each address and the affinity reagent is washed off the array before the next one is added via the next cycle. Integrated fluidics and imaging on instrument allow high resolution multi-cycle imaging of the addresses in the presence of the affinity reagents. Therefore, binding of affinity reagents to proteins produces a series of bind/no-bind outcomes for each protein, which can be used to infer the identity of the protein. Since there is only one protein per address, direct counting of the addresses can be used to quantify each protein identified in the sample.

Identifying the many different proteins in a human proteome, or other complex proteome, would require a prohibitively large number of highly specific affinity reagents. The present methods overcome this by using affinity reagents that bind short, linear epitopes (e.g., trimers) with moderate specificity, so that each affinity reagent binds many different proteins. While binding of a single affinity reagent is insufficient to identify any particular protein with these promiscuous affinity reagents, a series of affinity reagents can decode many different proteins. The detection of each new affinity reagent bound at each address across a growing number of cycles gradually narrows down the list of possible protein identities at each address (FIG. 1B).

In a typical single-molecule binding reaction format, binding is stochastic, as an affinity reagent will not always be observed to bind a protein containing its epitope (see Chang, et al., J Immunol Methods 378, 102-115 (2012), which is incorporated herein by reference). Furthermore, each affinity reagent may be observed to bind to off-target epitopes. Therefore, repeating the same series of single-molecule binding reactions multiple times will typically result in observation of multiple different binding patterns (FIG. 1C).

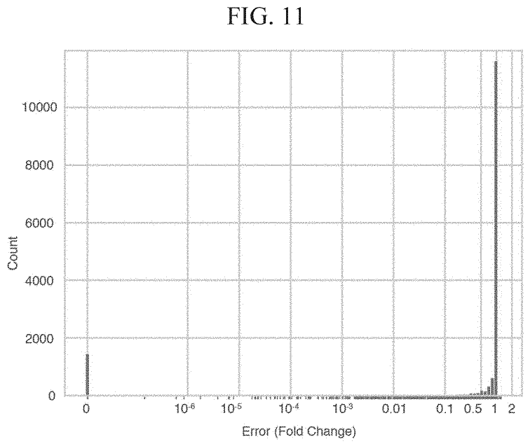

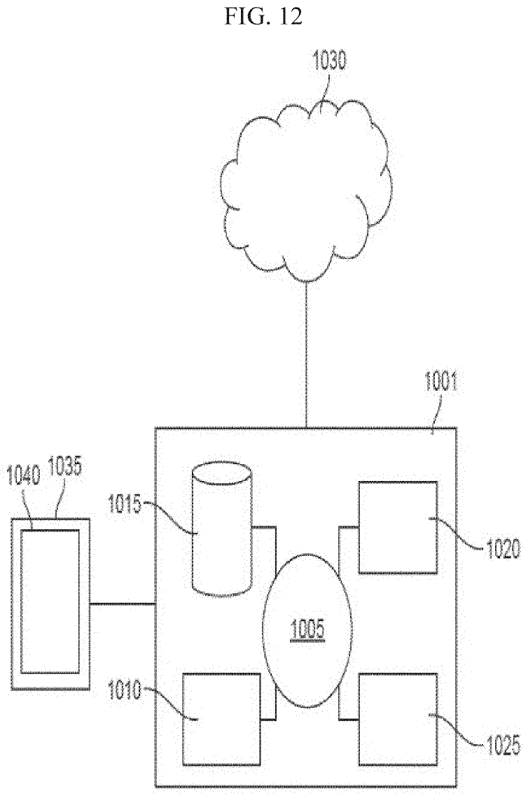

In view of this stochasticity, a binding model was devised whereby each affinity reagent binds with a primary probability to a protein containing one copy of its target epitope and with an equal or lower probability to a protein containing one copy of an off-target epitope. The rather low probability of 0.5 was initially selected for on-target binding to its primary epitope and 0.5 probability to binding to an off-target epitope because there are many factors that could prevent binding of an affinity reagent to its epitope, for example, residual or transient protein structure due to partial denaturation, presence of post-translational modifications, binding stochasticity or the like. To determine the affinity reagent selectivity that provides high coverage of the human proteome with a manageable number of different affinity reagents, affinity reagents with various target epitope lengths (dimer, trimer, or tetramer) and varying numbers of off-target epitopes were evaluated. As shown in FIG. 11D, the analysis showed that 100 affinity reagents would facilitate unique identification of 90% of the human proteome if each affinity reagent bound to a single trimer and 9 additional primary off-target trimers. In this scenario, each affinity reagent would bind about 23.700 of the proteins (N.B. the percentage being based on the number of unique protein sequences independent of variability in expression level for each protein) in the human proteome and about 24 binding events would be sufficient to identify a given protein on average (Table 1). Targeting tetramer epitopes would reduce the number of binding events but increase the number of affinity reagents sufficient to achieve similar coverage. Targeting dimer epitopes would allow for a similar number of affinity reagents, but it could be challenging to generate affinity reagents that recognize dimers independent of variability in the sequence surrounding the dimer. Therefore ‘trimer with 10 epitopes’ affinity reagent selectivity model was used for the present analyses.

TABLE 1
Affinity Reagent Characteristics

| | | | | Number of | | |
|---|---|---|---|---|---|---|
| | | | Number of | Affinity Reagents | % Landing Pads | % Landing Pads |
| | Number of | Number of | Affinity Reagents | Bound Per | Lit Per | Lit Per |
| Epitope | Epitopes per | Cycles for | Bound Per Protein | Protein | Affinity Reagent | Affinity Reagent |
| Type | Affinity Reagent | 90% Coverage | (mean) | (std dev) | (mean) | (std dev) |
| Dimer | 1 | 110 | 40.30728935 | 19.55502431 | 36.64299032 | 14.84719629 |
| Dimer | 2 | >2000 | 1102.618384 | 400.209814 | 55.1309192 | 15.78071197 |
| Trimer | 1 | 410 | 12.53679269 | 11.32908544 | 3.057754314 | 2.335865074 |
| Trimer | 2 | 250 | 14.44343958 | 12.2351679 | 5.777375834 | 3.636218605 |
| Trimer | 5 | 130 | 17.64635532 | 12.8720439 | 13.57411948 | 6.87105185 |
| Trimer | 10 | 100 | 23.70956264 | 14.70880367 | 23.70956264 | 10.00887062 |
| Trimer | 20 | 120 | 44.13743514 | 21.73416995 | 36.78119595 | 14.22655523 |
| Trimer | 25 | 150 | 62.95384235 | 28.56932135 | 41.96922823 | 14.86283342 |
| Trimer | 30 | 220 | 100.8525822 | 42.68880344 | 45.8420828 | 15.54123936 |
| Trimer | 40 | 690 | 361.7437608 | 136.1278615 | 52.426632 | 16.54646207 |
| Tetramer | 1 | >2000 | 3.552409192 | 3.996546384 | 0.17762046 | 0.215077644 |
| Tetramer | 2 | >2000 | 6.561551767 | 6.901374069 | 0.328077588 | 0.344402837 |
| Tetramer | 5 | 1350 | 10.71208302 | 10.60025175 | 0.793487631 | 0.730490262 |
| Tetramer | 10 | 760 | 11.43864591 | 10.92199034 | 1.505084988 | 1.22358617 |
| Tetramer | 20 | 430 | 12.63602669 | 11.43641187 | 2.938610857 | 2.210826628 |
| Tetramer | 25 | 370 | 13.20800593 | 11.69248829 | 3.569731333 | 2.62810432 |
| Tetramer | 30 | 320 | 13.50155671 | 11.71124183 | 4.219236471 | 3.073673392 |

It is also possible to use affinity reagents that are more specific, for example, binding to a single epitope or even a single protein. In some cases, multiple different affinity reagents can be combined to create a pool of affinity reagents that binds with apparent promiscuity. For example, a pool of 3 different affinity reagents that are indistinguishably detected from each other in a binding step would appear to promiscuously bind proteins targeted by the pool. By way of more specific example, a pool of 3 different affinity reagents may apparently bind at least 3 different proteins, a pool of 5 different affinity reagents may apparently bind at least 5 different proteins, a pool of 10 different affinity reagents could apparently bind at least 10 different proteins, etc.

In addition to having primary binding epitopes, affinity reagents are likely to bind other off-target epitopes, albeit with lower probability. A “biosimilar” affinity reagent model (see Methods section below) was used, whereby each affinity reagent had a “tail” of up to 20 additional secondary off-target epitopes, with binding probabilities proportional to the similarity of the off-target epitope to the target epitope. Using this model with target epitopes selected randomly from targets present in the human proteome, the decoding algorithm was able to uniquely identify about 98% of proteins in the human proteome (modeling a sample with one copy of each protein) with 300 cycles (FIG. 1E). Performance with less than 200 affinity reagents improved when using a greedy-selection algorithm (see Methods section below) to determine the optimal set of 300 trimer epitopes achieving high human proteome coverage with as few affinity reagent cycles as possible (FIG. 1E). This optimal set of epitopes was used for subsequent analyses.

To test whether the decoding strategy can be applied to proteomes from species other than humans, the same parameters were used with the same set of optimized affinity reagents to simulate analysis of proteomes from mouse, S. cerevisiae, and E. coli (FIG. 1F). Surprisingly, there was little difference between the species, indicating that while smaller proteomes are slightly easier to decode, the primary driver of decoding performance is protein sequence diversity. Therefore, despite the stochastic nature of single molecule binding, the decoding strategy has the potential to decode more than 90% of the proteome for a wide range of organisms.

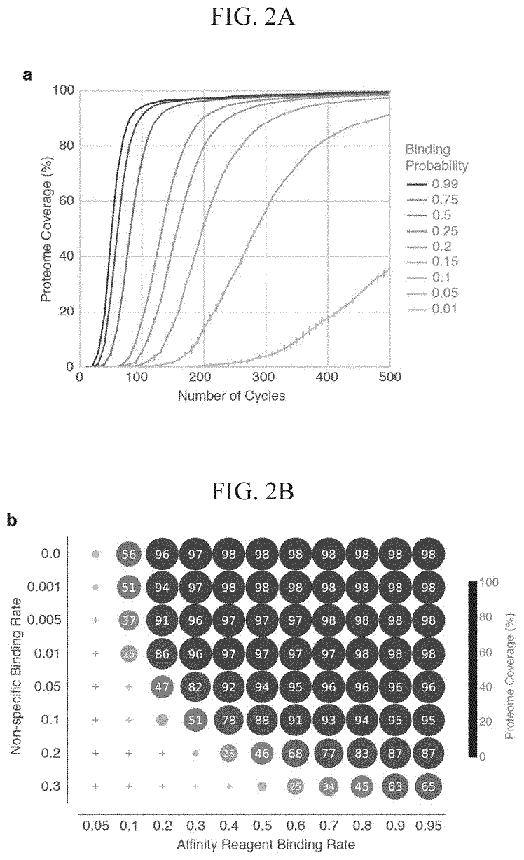

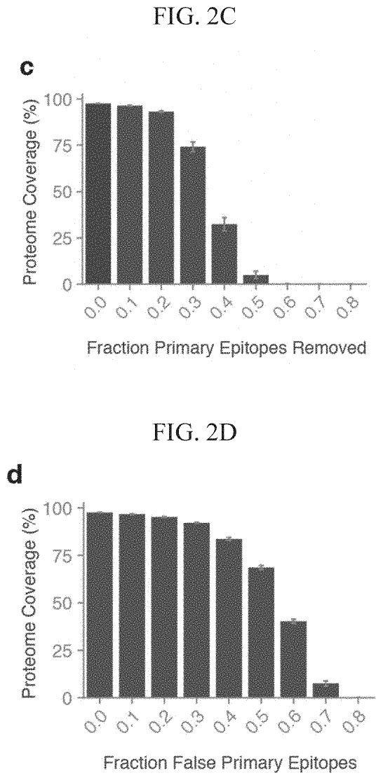

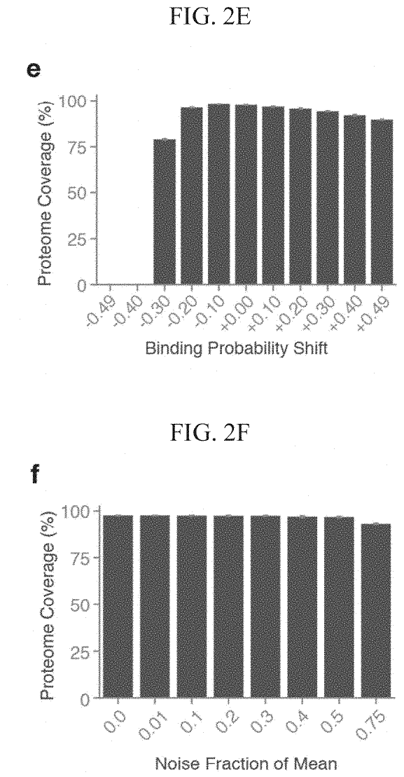

Potential experimental confounders were evaluated. A first scenario, in which the probability of affinity reagent to epitope binding is even lower than 0.5, for example due to poor binding affinity or kinetics, was considered. Even with a probability of 0.1, the decoding method achieved over 85% proteome coverage using 300 cycles (i.e. 300 different affinity reagents), although this dropped to about 55% when the binding probability was 0.05 (FIG. 2A). Options for increasing coverage include, for example, using more affinity reagents, multiplexing several affinity reagents in a single run (for example, using different fluorescent labels for each probe in a multiplexed set); running affinity reagents in replicate cycles to improve the chances of observing binding; increasing concentration of affinity reagents; increasing duration of the binding reaction; or attaching multiple copies of an affinity reagent to a scaffold such as a fluorescent particle or structured nucleic acid particle. Accordingly, the decoding method may be viable using affinity reagents across a range of binding probabilities, some of which are relatively low.

The effect of non-specific binding of an affinity reagent to the surface of an array at a location close enough to a protein address to create a false binding signal was evaluated. As demonstrated by FIG. 2B, assuming a binding probability of 0. 5, a non-specific binding rate of 0.05 or lower provided about 90% detection sensitivity. For subsequent analyses, a non-specific binding rate of 0.001 was assumed. If the rate proves to be higher experimentally, binding conditions (e.g. ionic strength, temperature, polarity, pH, osmolarity, concentration of affinity reagent or surface tension) can be adjusted to reduce non-specific binding. The same or different conditions can be used for each affinity reagent.

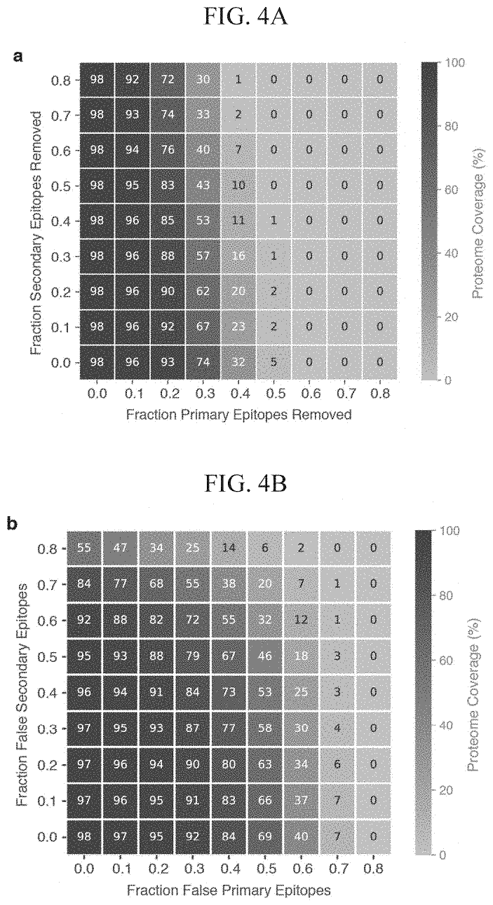

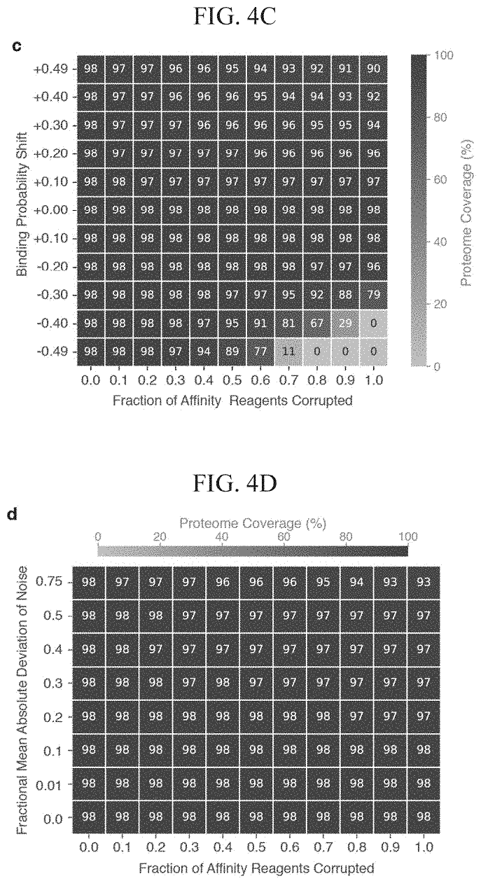

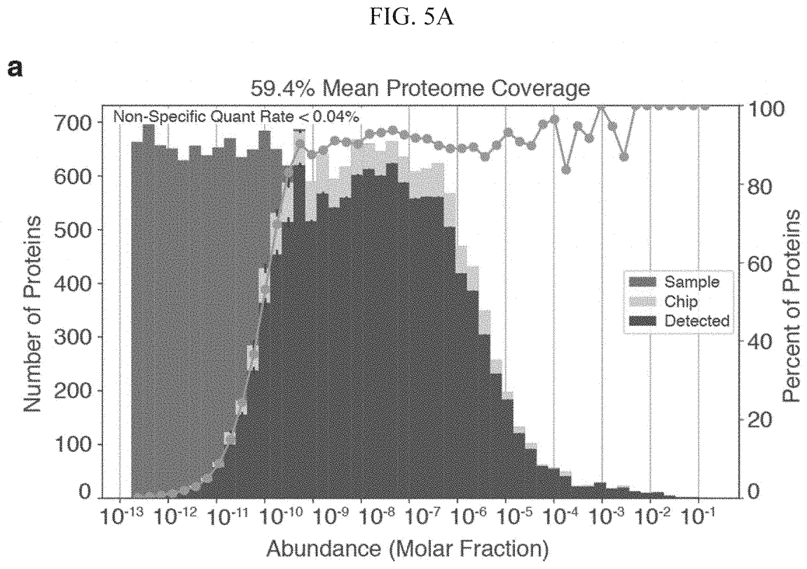

The impact of affinity reagent characterization (e.g. identification of target epitopes and off-target epitopes, and the respective binding probabilities) was also evaluated. Such characterization can be performed in a straightforward manner using traditional epitope mapping approaches (Beyer, et al., Science 318, 1888 (2007), which is incorporated herein by reference). Trimer epitopes may be “missed” during affinity reagent characterization, for example, if each affinity reagent binds an additional number of epitopes that the inference algorithm does not know about (FIG. 2C, FIG. 4A). However, the impact was small, so long as high probability (0.5) binding epitopes were not consistently missed. Proteome coverage remained above 92% if up to 20% of these epitopes were missed. Trimer epitopes may also be falsely identified as targets during affinity reagent characterization (FIG. 2D, FIG. 4B). The decoding method appeared to be robust to this type of error, as it achieved nearly 70% coverage even if half of all primary epitopes were incorrect. Given that the decoding method appeared to be more robust to having false positive epitopes than ‘missing’ epitopes in the affinity reagent model, the techniques used to characterize affinity reagents can be tuned more towards sensitivity rather than specificity to achieve improved results. Evaluation of the impact of consistent over- or under-estimation of affinity reagent to epitope binding probabilities indicated that the impact of such errors was small with the exception of large (>−0.2) underestimation of binding probability (FIG. 2E, FIG. 4C). The decoding method appeared to be highly robust to noisy affinity reagent characterization, indicating that affinity reagent characterization need not be perfect, and that the method will tolerate variability in affinity reagent binding characteristics that may arise from other potential experimental confounders such as temperature (FIG. 2F, FIG. 4D). In summary, the decoding method appeared to be robust to errors in the affinity reagent characterization.

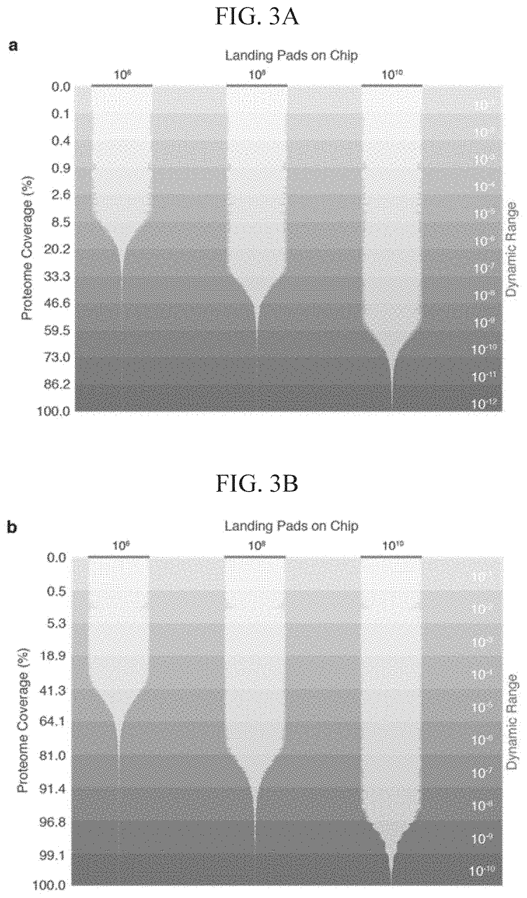

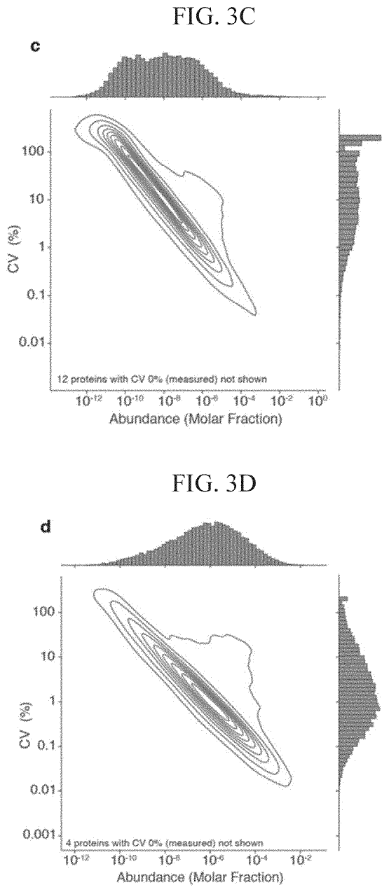

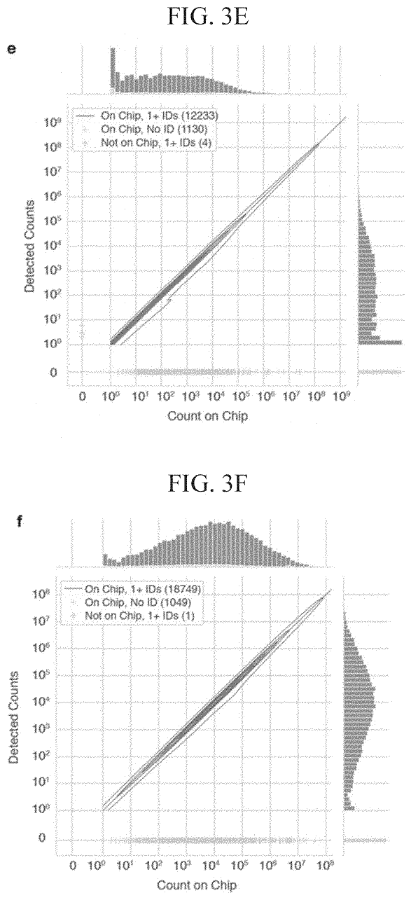

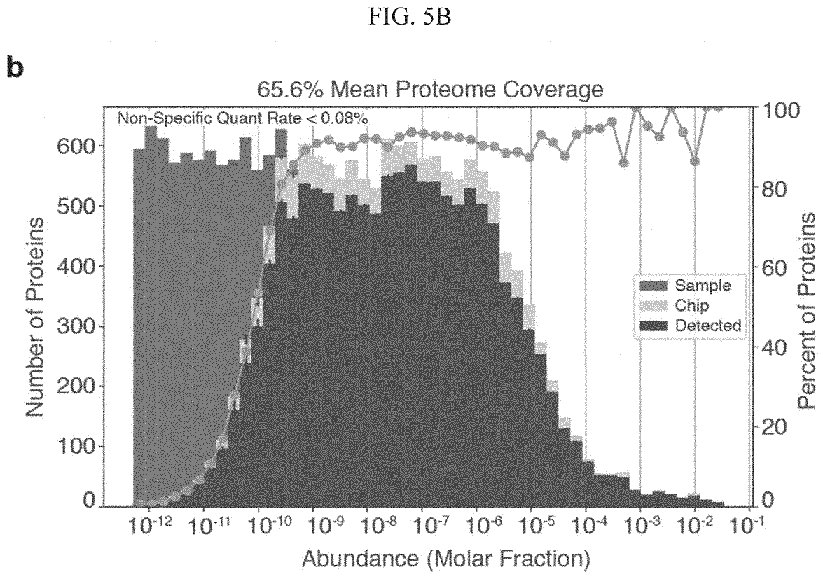

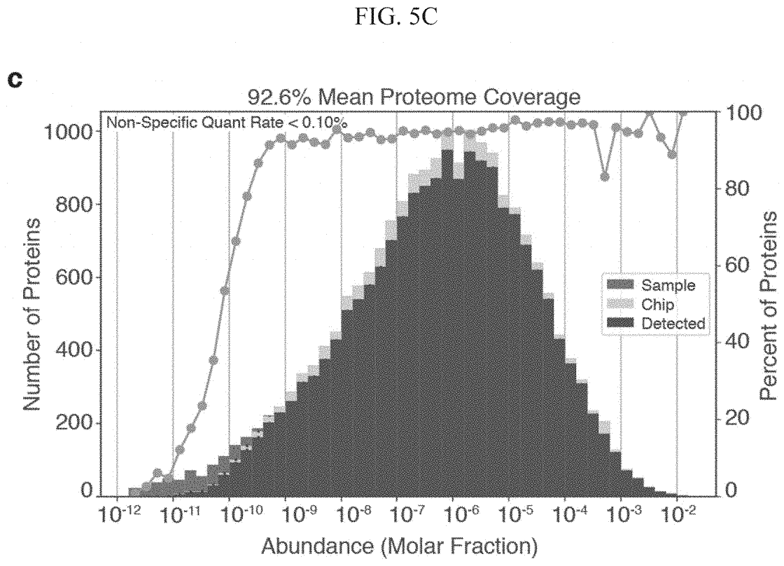

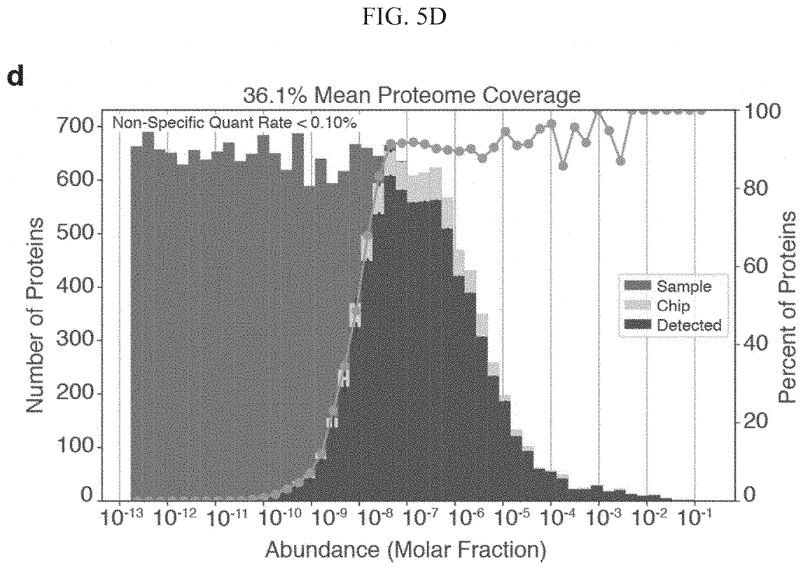

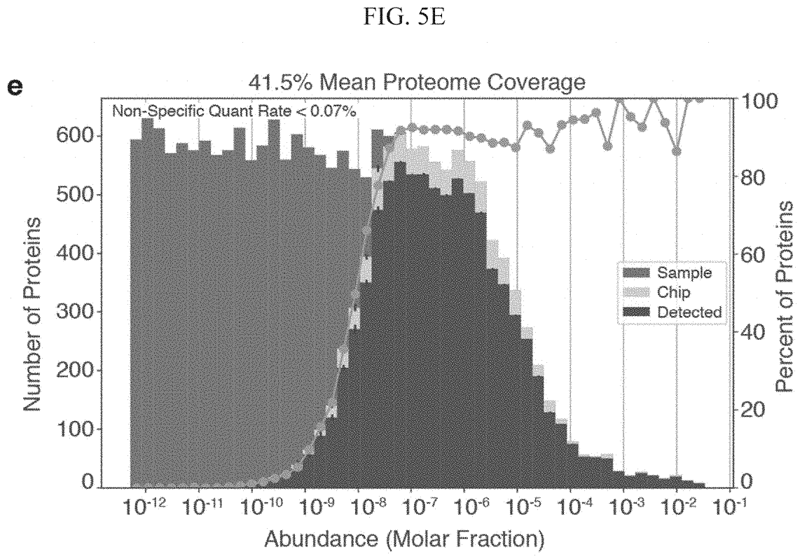

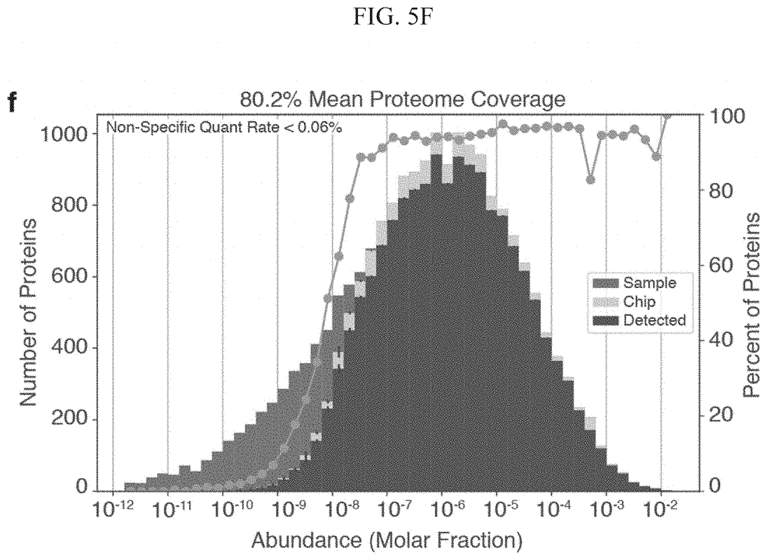

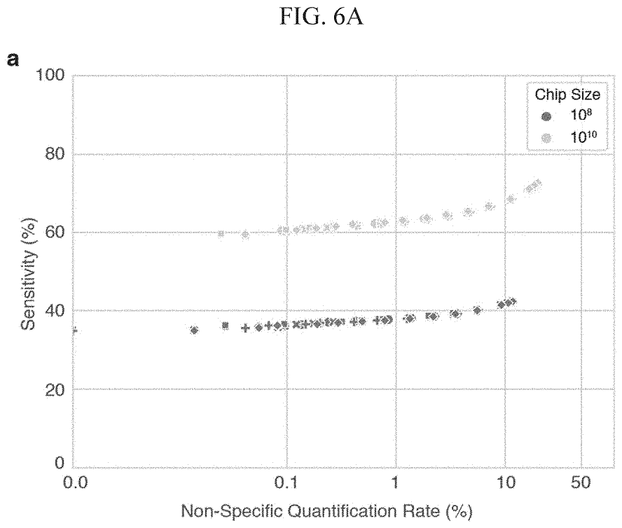

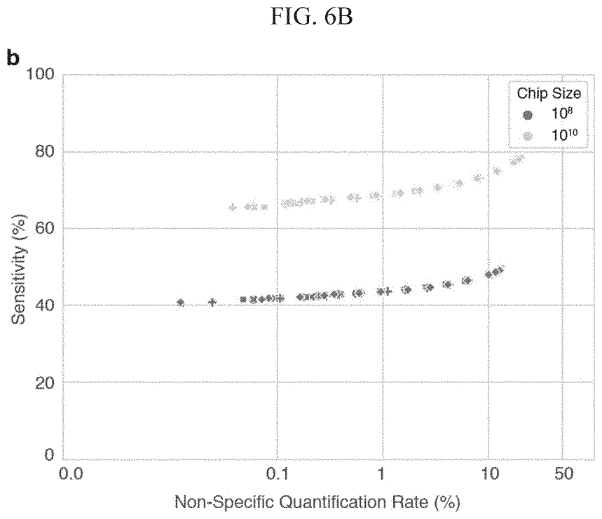

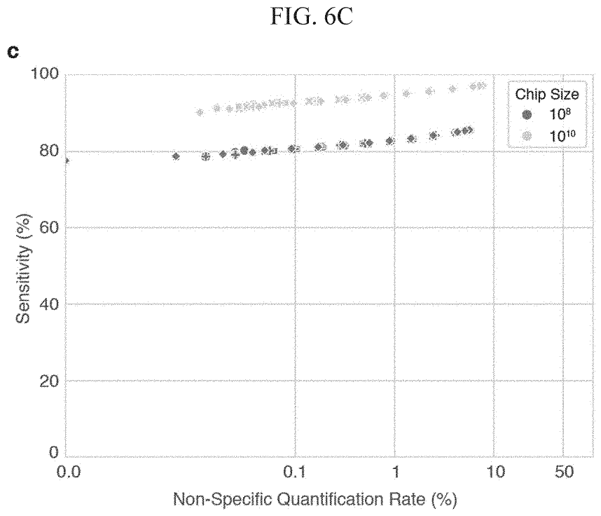

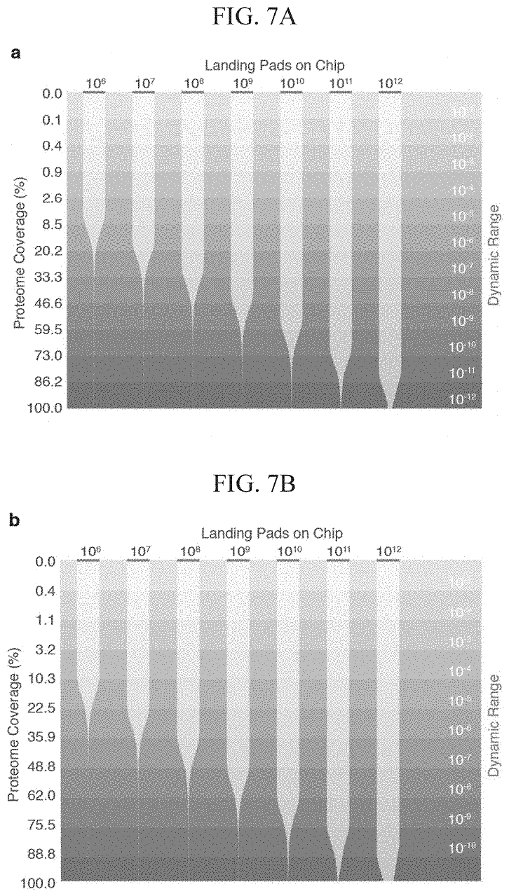

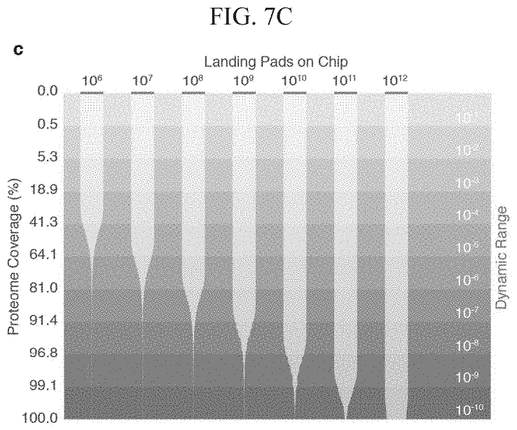

Blood plasma is a good example of one of the major challenges to proteomics, as plasma protein concentrations can vary by more than 12 orders of magnitude and typical mass spectrometry-based approaches typically only identify 8% of the proteome (see Anderson & Anderson, Mol Cell Proteomics 1, 845-867 (2002), which is incorporated herein by reference). To evaluate the theoretical performance of the protein decoding strategy, a simulation was run for assaying an un-depleted blood plasma sample with 300 affinity reagents on an array with 10⁶, 10⁸ and 10¹⁰ addresses. The simulation modeled running the same sample across five technical replicates. Some random noise in affinity reagent to trimer binding probability simulated variability in affinity reagent binding across replicates. On average, simulations executing the decoding algorithm with a 10¹⁰ address array demonstrated a detection dynamic range spanning>11.5 orders of magnitude ranging from the most abundant to the least abundant protein detected (FIG. 3A, FIGS. 5A-5F). The decoding method was able to quantify 59.4% of the 20,235 proteins in the modeled plasma sample. Almost all proteins were quantified with high specificity (FIGS. 6A-6C). More than 99.6% of the measured proteins had quantitative specificity >90% (i.e., >90% of identifications of the protein were true positives). Proteins within the top 9 orders of magnitude dynamic range were detected with 90% consistency. Bias in identifiability that correlated with protein concentration was not observed. Overall, 90% of proteins deposited on the array were detected, indicating that the ability to deposit low concentration proteins on the array, rather than the ability to decode proteins, is the primary limiter of dynamic range. Modeling suggests that increasing the number of addresses to 10¹¹ or 10¹² would increase identification of proteins deposited on the array from 66% to 79% and 92%, respectively (FIGS. 7A-7C)

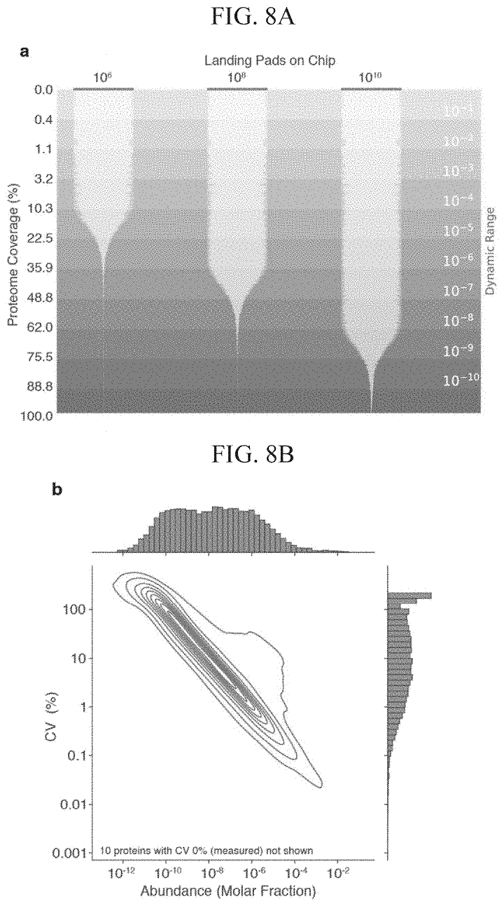

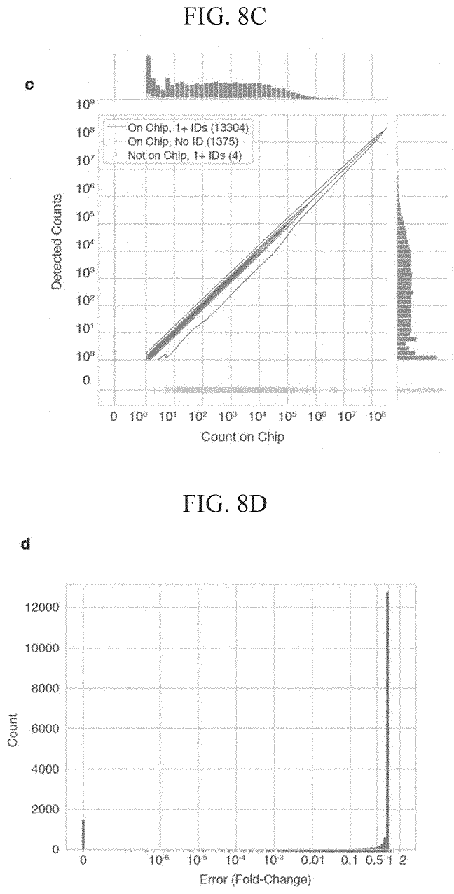

Experimentally, the dynamic range could be compressed by depleting the most abundant proteins in a plasma sample, for example, using an affinity column. A plasma sample modeled with 99% depletion of the top 20 proteins had 65.7% proteome coverage on average (FIGS. 8A-8D). Coverage was substantially higher (92.6%) when modeling a HeLa cell-line sample, which has a lower dynamic range (detection spanned 9.5 orders of magnitude) (FIG. 3B).

In all samples, some proteins with relatively high abundance were not detected because detectability is not just a factor of abundance but also sequence similarity. If the sequence of a protein is very similar to another protein in the database, it can be difficult for the decoding algorithm to generate confident identifications for these proteins. More selective affinity reagents can be used to detect these more difficult targets.

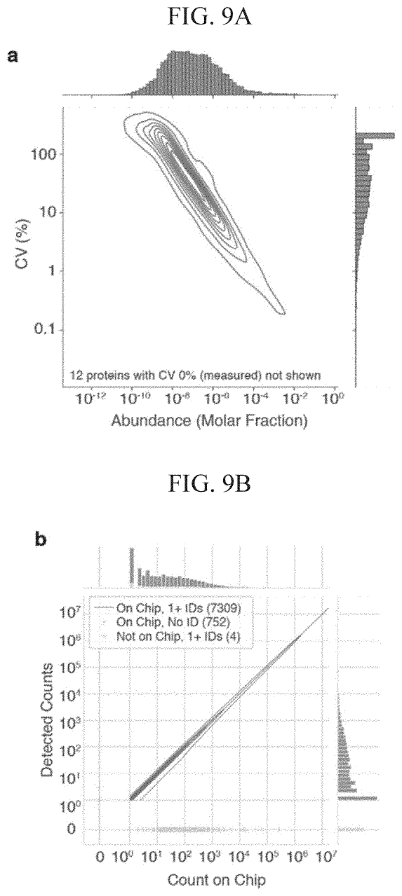

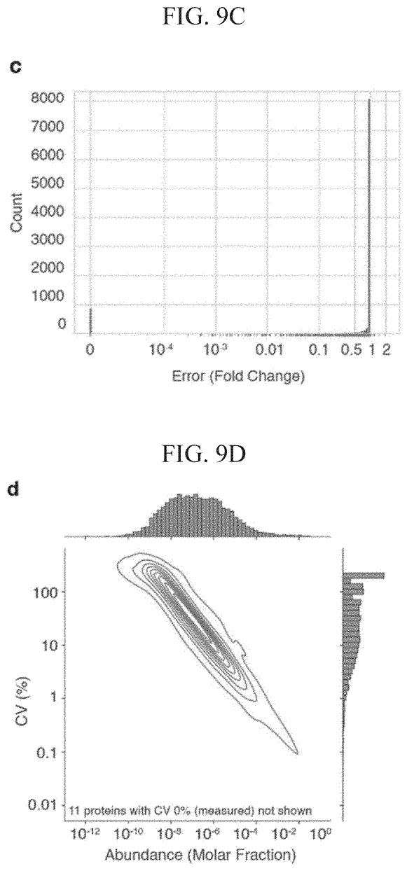

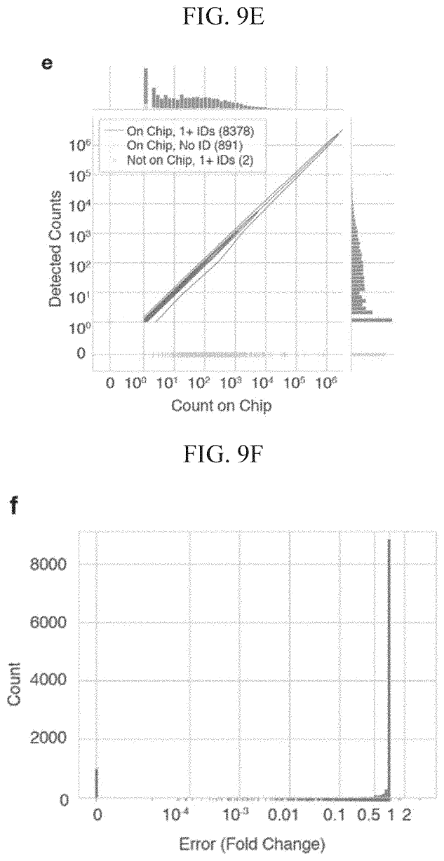

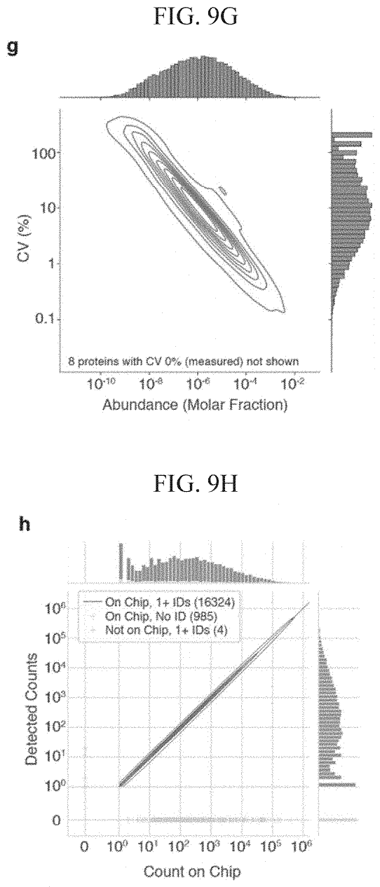

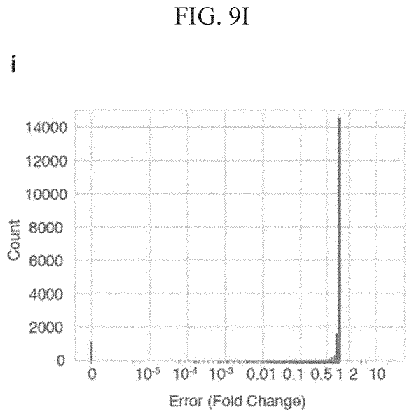

A strategy to increase throughput would be to use an array of 10⁸ protein addresses for each proteome sample (e.g., multiplexing multiple proteome samples on an array or running multiple smaller arrays in parallel). In this situation, the low abundance proteins became undetectable resulting in a compressed dynamic range spanning 7.5 orders of magnitude (for proteins detected consistently) in plasma but with high coverage within that range (FIGS. 9A-9I).

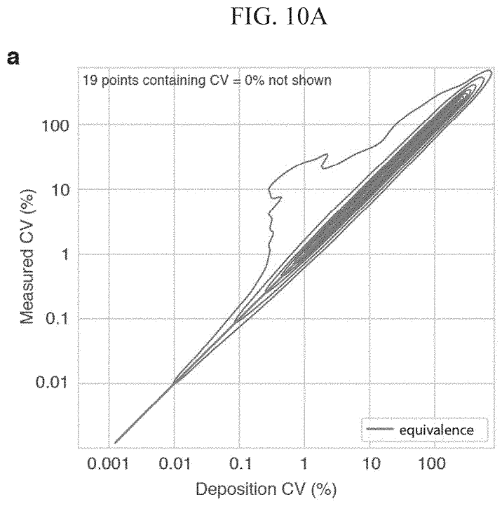

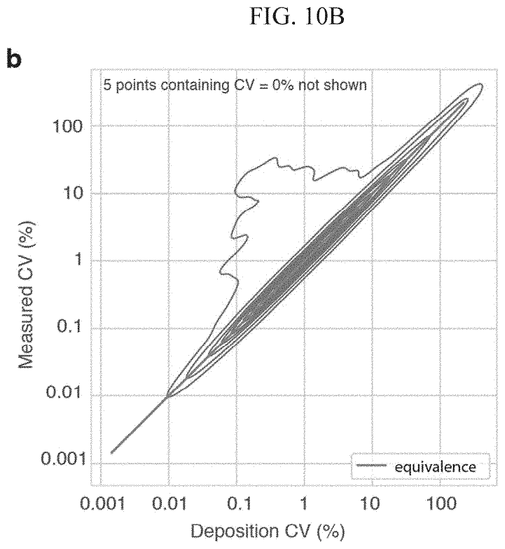

Measurement reproducibility was assessed across the five technical replicates of the modeled blood plasma and HeLa samples (FIGS. 3C & 3D). The coefficient of variation (CV) was <10% for medium to high abundance proteins. Proteins within the top 5 orders of magnitude in terms of abundance in the plasma sample generally had CV<1%. As modeled, the contributors to irreproducibility were stochastic variation in affinity reagent binding and protein deposition as well as variation in affinity reagent binding characteristics. While these estimates do not consider many factors of experimental variability such as sample preparation and biological variability, they demonstrate the potential of the analytic platform and decoding algorithm to contribute minimal variation relative to more common sources of variation. In fact, the CV observed in measurement counts was not much different from the CV of the actual counts, indicating that reproducibility of measurements can be improved by increasing throughput (FIGS. 10A & 10B).

Detected protein counts correlated with the number of proteins modeled on the array (FIGS. 3E & 3F). 76% of plasma proteins had a fold change error in detected counts relative to counts on array within +/−10% (FIG. 11). In some cases, proteins with only a single copy on a chip were detected. Some proteins were substantially under-counted due to sequence similarity to other proteins in the sequence database. The linear nature of detection count vs. counts on array indicated that dynamic range can be extended further by expanding the array to 10¹¹ addresses or evaluating a sample across multiple arrays.

In conclusion, the results presented in this example provide a theoretical foundation for a single-molecule protein identification method that is proteome invariant and can be used to analyze the entire human proteome in a single experiment. It has important advantages over other proteome analysis methods. It is unique amongst the emerging single-molecule peptide sequencing methods in taking a non-destructive affinity reagent approach, rather than a chemically intensive or cleavage-based sequencing approach. It is robust to false negatives (i.e. failure of an affinity reagent to bind its epitope) and is optimized for non-specific affinity reagents. Therefore, the decoding method turns a common weakness of affinity-based proteomics approaches into a strength. The decoding method is scalable to full proteome quantification and, unlike mass spectrometry, is capable of quantification over a wide dynamic range. By using intact proteins, the decoding method avoids the loss of information (such as proteoforms) that limits approaches that are based on detecting peptide fragments of proteins, and partially mitigates the dynamic range challenge, as sample complexity is decreased by approximately two orders of magnitude. If successfully implemented experimentally, the decoding method will provide a user-friendly, rapid, ultrasensitive, and reproducible method to analyze and quantify proteomes, even from single cells. The decoding method is expected to open a path to countless new opportunities in scientific discovery, not only in basic research but also in clinical research, including molecular diagnostics and biomarker discovery.

The simulations set forth in this example, indicate the potential power of implementing a sensitive and rapid imaging platform. As the dynamic range of the exemplified decoding method is directly related to the number of intact protein molecules measured, a particularly useful detection system will have rapid imaging and cycle speed. Preliminary estimates suggest that, with 300 affinity reagents and cycle times of approximately 10 minutes, it will be possible to profile ten-billion protein molecules within about a day. A successful experimental implementation of the decoding method will provide a user-friendly, rapid, ultrasensitive, and reproducible method to analyze and quantify proteomes, even from single cells. It would open a path to countless new opportunities in scientific discovery, not only in basic research but also in clinical research, including molecular diagnostics and biomarker discovery.

### Methods

### Protein Sequence Databases

Protein sequence databases were downloaded from Uniprot (www.uniprot.org). For each species, the “reference” proteome was selected by including “reference:yes” in the search query string for proteomes. The reference proteome was then filtered to only include Reviewed (Swiss-prot) sequences (query string “reviewed:yes”). The sequence data was then downloaded in uncompressed .fasta format (canonical sequences only). Specific proteomes and filter strings used were:
E. coli (strain K12): reviewed:yes AND organism:“Escherichia coli (strain K12) [83333]” AND proteome:up000000625 (downloaded 6/30/2021)
S. cerevisiae (s288c): reviewed:yes AND organism:“Saccharomyces cerevisiae (strain ATCC 204508/S288c) (Baker's yeast) [559292]” AND proteome:up000002311 (downloaded 6/30/2021)
M. musculus (c57bl): reviewed:yes AND organism:“Mus musculus (Mouse) [10090]” AND proteome:up000000589 (downloaded 6/30/2021)
H. sapiens: reviewed:yes AND organism:“Homo sapiens (Human) [9606]” AND proteome:up000005640 (downloaded 7/6/2021)

The proteomes were further processed to remove any duplicated sequences and any sequences not entirely composed of the 20 canonical amino acids. Further, sequences of length 30 or less were removed from each FASTA.

### Modeling Affinity Reagent to Protein Binding

An affinity reagent targeting epitopes of length k (e.g. for a trimer, k=3) was modeled by assigning a binding probability θ to each unique target epitope j of length k recognized by the reagent. Further, a protein non-specific binding rate was assigned p_{nsbepitope} representing the probability of the affinity reagent binding to any epitope in a protein non-specifically. Given the primary sequence for a protein of length M, the probability of an affinity reagent binding to the protein was computed as follows:

First the probability of a specific binding event happening was computed:

$${p}_{specific} = 1 - {\prod }_{j = 1}^{8000} {( 1 - {θ}_{j} )}^{{x}_{j}}$$

with:
X: the count of each epitope j in the protein sequence
   X={x₁, x₂, x₃ . . . } with xⱼ∈ [symbol] *
θ: the binding model parameters. A vector of probabilities of the affinity reagent binding to each recognized epitope
   θ={θ₁, θ₂, θ₃, . . . } with 0<θⱼ≤1.

Next, the probability of a non-specific protein binding event happening was computed:

p_{nonspecific}=1−(1−p_{nsbepitope})^{M-k+1}

with:
p_{nsbepitope}: the probability of the affinity reagent non-specifically binding to any epitope in the protein
   0≤p_{nsbtrimer}≤1
M: the length of the protein sequence
k: the length of the linear epitope(s) recognized by the affinity reagent.

The probability of the affinity reagent binding to the protein and generating a detectable signal was the probability of 1 or more specific or non-specific binding events occurring:

p_{proteinbind}=1−(1−p_{specific})*(1−p_{nonspecific})

When noted, the probability of binding to each protein was adjusted to account for additional random surface non-specific binding (NSB). That is, binding of an affinity reagent to the array close enough to the protein address to generate a false-positive binding event. The prevalence of surface NSB is defined as a probability 0≤p_{surfacensb}<1 of such a surface NSB event occurring during the acquisition of a single affinity reagent measurement at a single protein location on the array. The adjusted probability of a protein binding event taking into account surface NSB was:

p_{adjustedbind}=1−(1−p_{proteinbind})*(1−p_{surfacensb})

### Biosimilar Affinity Reagent Model

Unless specifically noted, affinity reagents were modeled using a “biosimilar” model. In this model, an affinity reagent targets a specific epitope which it binds with probability 0.5. The affinity reagent also binds nine additional primary off-target epitopes with probability 0.5 that are biosimilar to the targeted epitope. Biosimilar targets were selected by computing a pairwise similarity score of the target epitope to every other possible epitope of the same length. The similarity score was computed by summing up the BLOSUM62 similarity between the pair of residues at each sequence location. For example, if computing the similarity of a trimer SLL with trimer YLH, the score would be BLOSUM62(S,Y)+BLOSUM62(L,L)+BLOSUM62(L,H). With all pairwise similarity scores computed, the top nine most similar epitopes to the target were selected as the primary off-target epitopes. In the case of a tie where multiple potential off-target epitopes have the same score, a random epitope was selected. In addition to the target epitope and four off-target epitopes, up to 20 additional secondary biosimilar off-target epitopes of lower binding probability were added to the affinity reagent. The 20 secondary off-target epitopes bind to the next 20 most biosimilar epitopes beyond the ones already included in the affinity reagent model. These 20 additional epitopes have a probability computed as:

b*(1.5^{ot-ss})

with:
b=binding probability of the affinity reagent to its target,
ot=BLOSUM62 similarity score between affinity reagent target and this off-target epitope,
ss=BLOSUM62 similarity score between affinity reagent target and itself.
If any of these additional off-target epitopes had binding probability that was less than the affinity reagent epitope non-specific binding rate, it was not included. The epitope non-specific binding probability was set at 2.45×10⁻⁸.

### Simulation of Stochastic Affinity Reagent Binding

To simulate binding of a series of affinity reagents to a single protein, the binding probability θᵢ of each affinity reagent i to the protein was first determined using the methods described in the Modeling Affinity Reagent to Protein Binding section above. To simulate the outcome of the binding for each affinity reagent, a single random draw was taken from the Bernoulli distribution parameterized by θᵢ. An outcome of 1 is binding, an outcome of 0 is no binding.

### Protein Decoding

### Overview

The protein decoding algorithm analyzed a series of affinity reagent binding measurements acquired on an extant protein and determined the most likely identity of that protein among a set of candidates. The most likely protein identity was the one most compatible with the observed binding measurements. This compatibility was determined based on a binding model for each affinity reagent in the experiment which were used to estimate how likely each affinity reagent was to bind each potential protein. A strong candidate protein was one where most of the observed binding events were consistent with affinity reagents likely to bind that protein. A weak candidate protein will have many instances where binding is observed for affinity reagents that are not expected to bind the candidate. The strongest candidate protein was deemed the most likely identity for the extant protein and confidence in this identification was computed as a relative measure of the compatibility of the most likely protein compared to all the other candidates.

### Inputs

The inputs to the decoding algorithm were:
Binding data: D=[d₁, d₂, d₃ . . . d_{N}] with d∈{0(nobind),1(bind)}. A sequence of binding measurements, one for each affinity reagent to an extant protein.
A sequence database of length M containing the primary sequence and name of each potential protein that may be present in the sample (e.g., the human protein sequence database described in section Protein Sequence Databases above)
A parameterized binding model for each of the N affinity reagents used in the experiment (see section Modeling Affinity Reagent to Protein Binding above).
An optional surface non-specific binding rate (r) describing the probability of a surface non-specific binding event happening at any one address in any given cycle.

### Binding Probability Calculations

An M×N binding probability matrix B was computed describing the probability of each affinity reagent binding to every possible candidate protein with an entry in the matrix b_{i,j} being the probability of affinity reagent j binding to candidate protein i. These probabilities were computed using the methods described in the Modeling Affinity Reagent to Protein Binding section above.

Next, the M×N matrix U with adjusted non-binding probabilities for each affinity reagent to each protein was computed as follows:
Compute S=[s₁, s₂, s₃, . . . s_{M}] where sᵢ=proteinᵢ length−2.
Compute F=[f₁, f₂, f₃, . . . f₈₀₀₀] the relative frequency of every possible unique trimer among the set of all candidate protein sequences where:

$${f}_{p} = \frac{trime {r}_{p} frequency}{{\sum }_{q = 1}^{8000} trime {r}_{q} frequency}$$

Compute A=[a₁, a₂, a₃, . . . a_{N}] the vector of average trimer non-binding probabilities for the affinity reagents. A value aⱼ in A is the probability of the affinity reagent not binding to a trimer, averaged over all 8000 trimers and weighted by the relative frequency of each trimer in the candidate protein database aⱼ=Σₚ₌₁⁸⁰⁰⁰ fₚ(1−t_{p,j})(1−cⱼ) where t_{p,j} is the probability of affinity reagent j binding to trimer p and cⱼ is the probability of a non-specific protein binding event happening for affinity reagent j.
Compute U where u_{i,j}=aⱼ^{s}^{ᵢ}(1−r) is the adjusted probability of affinity reagent j not binding to protein i (r is the surface NSB rate).

Adjusted non-binding probabilities were computed in this manner (as opposed to U=1-B) to avoid any single non-binding event having an outsized impact on a protein. The rationale was that there are numerous difficult to predict reasons why an affinity reagent may not bind to a specific epitope (e.g., protein structure, post-translational modifications) and so the total number of non-binding events should be considered more than the specific identity of the observed non-binding events.

### Decoding

A vector of likelihoods for each protein in the candidate database was computed by multiplying the likelihoods of each observed binding event:

$$\cdot L = [ {ℒ}_{1} , {ℒ}_{2} , {ℒ}_{3} … {ℒ}_{M} ] where :$$

$${ℒ}_{i} = {\prod }_{j = 1}^{j = N} ( {d}_{j} {b}_{i , j} + ( 1 - {d}_{j} ) {u}_{i , j} )$$

The protein of highest likelihood was selected (if there was a tie for top protein, one of the top proteins is selected randomly):
ID=argmax(L)

The probability of the ID being correct is the likelihood of the top protein divided by the sum of the likelihood of all other proteins:

$$\cdot probability = \frac{{ℒ}_{ID}}{{\sum }_{i = 1}^{i = M} {ℒ}_{i}}$$

The protein ID and probability are the outputs for the decoding process performed on a single extant protein.

### Calculation of Proteome Coverage

To compute proteome coverage, a set of affinity reagents was defined as in the Modeling Affinity Reagent to Protein Binding section above. Binding of the affinity reagents was simulated for each protein (see the Simulation of Stochastic Affinity Reagent Binding section above) in the human proteome as defined in the Protein Sequence Databases section above. The binding data was then passed to the decoding algorithm along with the definition of the affinity reagents, and the FASTA sequence database. The output of the decoding algorithm was a single protein identification for each simulated protein and an estimated probability of that identification being correct. To compute the fractional coverage, the number of proteins identified above a true/false discovery rate threshold of 1% (see the Computing and Thresholding on False Discovery Rate section below) was divided by the total number of proteins simulated. The percent coverage was computed by multiplying fractional coverage by 100. This method was applied for all analyses except for modeling of cell, plasma, and depleted plasma samples which use the method described in the Quantitative Statistics section below.

### Computing and Thresholding on False Discovery Rate

Given a list of decoded protein identities (protein identity and associated probability), the false discovery rate was computed by first annotating each protein identification as correct or incorrect based on its match to the true identity of that protein in the simulation. For each unique identification probability in the list, the false discovery rate (FDR) was computed as the fraction of proteins at that probability or lower that were incorrectly identified. To threshold on false discovery rate, the lowest probability score threshold with FDR less than the desired FDR was determined. Identifications at this probability score or higher satisfied the FDR criterion and were considered “identified” at the desired FDR threshold.

### Demonstration of Stochastic Binding

Stochastic binding of a sequence of 10 affinity reagents to protein EGFR was simulated six times (FIG. 1C). Affinity reagents with binding sequence present in EGFR have a 0.5 probability of binding and those without a binding sequence in EGFR have 0 probability of binding. Binding was simulated as described in the Simulation of Stochastic Affinity Reagent Binding section above.

### Evaluation of Affinity Reagent Requirements for Efficient Decoding

Affinity reagents with various target epitope lengths (2, 3, or 4 i.e., dimer, trimer, tetramer, respectively) with varying numbers of primary off-target epitopes were modeled. In each case, the target binding probability was 0.5. “Number of Epitopes per Affinity Reagent”=1 represents affinity reagents targeting a single epitope, with no primary off-target epitopes. Other scenarios were modeled with the affinity reagents having some number of primary biosimilar (see the Biosimilar Affinity Reagent Model section above) off-target epitopes. For example, an affinity reagent labeled as targeting ‘5’ epitopes has binding affinity for its target and four primary off-target sites. Affinity reagents did not have any secondary off-target epitopes (see Biosimilar Affinity Reagent Model section above). The targets of affinity reagents were selected randomly from targets present in the proteome. There was no requirement for off-target binding epitopes being present in the proteome.

To determine the number of affinity reagents required to achieve 90% coverage of the proteome, binding of an excess of affinity reagents (i.e., more than required for 90% coverage) was simulated to each protein in the proteome. For any number of affinity reagents N, the proteome coverage was computed using the first N affinity reagents in the set. The number of affinity reagents required to achieve 90% proteome coverage was the lowest N with coverage at or exceeding 90%. The values of N tested were in increments of 10.

With the number of affinity reagents (N) required for 90% coverage computed, the number of binding events observed for each simulated protein was recorded, and the mean of these values reported as the “Average Number of Binding Events per Protein”. Additionally, the percent of proteins generating a binding event for each affinity reagent was recorded, and the mean of these values was reported as the “Percent of Proteins Bound Per Affinity Reagent”.

### Selection and Evaluation of Optimal Affinity Reagent Trimer Targets

The standard biosimilar affinity reagent model (see Biosimilar Affinity Reagent Model section above) was used in this analysis with trimer-targeting affinity reagents. One set of ‘optimal’ affinity reagent targets was computed by using a greedy-selection algorithm to estimate the optimal set of 300 targets to achieve high proteome coverage with as few affinity reagents as possible. Additionally, 20 sets of 300 targets were selected randomly among trimers present in the proteome (excluding any trimers containing a cysteine). Proteome coverage for each of the 21 affinity reagent sets was evaluated as described in the Calculation of Proteome Coverage section above. Proteome coverage was also evaluated for multiple first-N reagent subsets of each affinity reagent set to evaluate scaling of proteome coverage with number of affinity reagents used.

The optimal set of trimer targets was chosen as set forth below:
1. Initialize an empty list of selected affinity reagents (AR).
2. Initialize a set of candidate ARs (e.g., a collection of 6,859 ARs, each targeting a unique trimer without a cysteine in it).
3. Select a set of protein sequences to optimize against (e.g., all human proteins in the UniProt reference proteome).
4. Repeat the following until the desired number of ARs has been selected:
   a. For each candidate AR:
      i. Simulate binding of the candidate AR against the protein set.
      ii. Perform decoding for each protein using the simulated binding measurements from the candidate AR and the simulated binding measurements from all previously selected ARs.
      iii. Calculate a score for the candidate AR by summing up the probability of the correct protein identification for each protein determined by protein inference.
   b. Add the AR with the highest score to the set of selected ARs, and remove it from the candidate AR list.

### Evaluation of Proteome Coverage in Multiple Organisms

Proteome coverage was assessed for four different organisms using the 300 affinity reagents targeting the optimal trimer set (see Selection and Evaluation of Optimal Affinity Reagent Trimer Targets section above) designed against the human proteome. Sequence databases for each organism are described in the Protein Sequence Databases section above. For each organism, binding was simulated using an affinity reagent epitope binding affinity of 0.5 for each affinity reagent against each protein in the sequence database for that organism. The binding data were then decoded using the appropriate sequence database for the organism and proteome coverage assessed as described in the Calculation of Proteome Coverage section using various first-N subsets of the 300 affinity reagent set. For example, to compute coverage at 100 affinity reagents for a given organism, only data from the first 100 of the 300 affinity reagents total were considered when decoding.

### Application of Noise to Affinity Reagent Binding Probabilities

A method was devised to model random perturbations in affinity reagent binding characteristics. The method applied random “noise” to the trimer (or other short linear epitope) binding probabilities while maintaining probabilities bound between 0 and 1. Given a binding probability p a perturbed probability was determined by drawing a sample from the distribution:

Φ(Φ⁻¹(p)+ [symbol] (0,σ²))

where:
[symbol] is the normal distribution,
σ² is a parameter used to tune the severity of the perturbation, and
ΦP is the cumulative distribution function of the standard normal distribution.

The parameter σ² was set such that the mean absolute deviation (MAD) of the distribution divided by the trimer probability p was equal to a desired target. This tuning parameter will be referred to as the “fractional MAD”. The fractional MAD was used to tune the noise due to its conceptual similarity to the coefficient of variation (standard deviation divided by mean) often used to describe measurement noise or reproducibility for normally-distributed measurements.

A numerical approximation method was used to find the value of σ² for a probability p that results in the desired fractional MAD. First, given p and the desired fractional MAD, the target MAD was computed as fractional MAD*p. A function optim is defined which, given p the target MAD, and a proposed σ² value generates 10,000 random samples from the noise distribution parameterized by p and σ² and returns the absolute value of the difference between the MAD of the 10,000 random samples and the target MAD. The minimize_scalar function from the scipy Python package is used to estimate the value of σ² which minimizes this function. This process is repeated 50 times, and the median optimal σ\hat{ }2 among the 50 trials is taken as the appropriate value to generate a noise distribution with the desired MAD.

### Modeling of Experimental Confounders

### Poor Binding Affinity

Proteome coverage (see Calculation of Proteome Coverage section above) was assessed using the 300 affinity reagents targeting the optimal trimer set (see Selection and Evaluation of Optimal Affinity Reagent Trimer Targets section above) binding to each unique protein in the human proteome (FIG. 2A). However, the affinity reagents were modeled with a variety of target epitope binding rates ranging from 0.01 to 0.99 to simulate varying affinity reagent binding affinity. Proteome coverage was assessed as described in the Calculation of Proteome Coverage section using various first-N subsets of the 300 affinity reagent set to model the relation between number of affinity reagents used and proteome coverage. Binding simulation and decoding were repeated five times to generate replicate analyses.

### Non-Specific Binding to Array Surface

Proteome coverage was assessed with varying combinations of affinity reagent binding affinity and non-specific binding rate. In every case, 300 affinity reagents targeting the optimal trimer set (see Selection and Evaluation of Optimal Affinity Reagent Trimer Targets section above) were used. However, the affinity reagents were modeled with a variety of target epitope binding rates ranging from 0.05 to 0.95 to simulate varying affinity reagent binding affinity and also varying surface non-specific binding ranging from 0 to 0.3. After modeling binding with surface NSB, proteome coverage was computed as described in the Calculation of Proteome Coverage section above.

### Missed Trimers During Affinity Reagent Characterization

Binding measurements for each of the set of optimal affinity reagents (see Simulation of Stochastic Affinity Reagent Binding section above) were generated against each of the proteins in the human FASTA database (see Protein Sequence Databases section above) with a surface NSB rate of 0.1% (see Non-Specific Binding to Array Surface section above). Prior to decoding the binding measurements to generate protein IDs, the affinity reagent models were corrupted by removing a fraction of primary epitopes. Such a corruption could occur in an experimental setting, for example, if the method used to determine the epitopes that an affinity reagent binds to missed some number of epitopes. The corrupted affinity reagent models were used when decoding the binding measurements to generate protein IDs and were expected to reduce decoding performance. The severity of the corruption was modulated by adjusting the percentage of primary epitopes that were missed. To model 20% of primary epitopes being missed, a random 20% of the primary epitopes (among all affinity reagents collectively) were selected for removal. Because the optimal affinity reagents have ten primary epitopes, this means that on average, two primary epitope was missed in each affinity reagent, although some may have more than one removed and others may have none removed due to random chance. In some analyses, a percentage of secondary epitopes were also removed in a similar manner.

### False Identification of Trimer Epitopes During Affinity Reagent Characterization

Similar to the Missed Trimers During Affinity Reagent Characterization section above, binding of affinity reagents to proteins in the proteome was simulated with surface NSB 0.1% and affinity reagent models were corrupted prior to decoding. For this analysis, false positive epitopes were added to the affinity reagents prior to decoding. This simulates a scenario where the method used to characterize the epitopes bound by each affinity reagent falsely identifies some number of trimer epitopes which the affinity reagent does not bind to. The severity of the corruption was modulated by adding false primary epitopes such that the complete set contained a specific percentage of false epitopes. For example, 20% false epitopes means that false primary epitopes were added until 20% of the primary epitopes among the affinity reagent set were false. The extra epitopes were randomly distributed among the affinity reagents. The trimer identities of the extra epitopes were selected randomly with replacement. In some analyses, secondary epitopes were also impacted by corruption. Any added secondary epitopes must not match an existing or added primary epitope. For example, an affinity reagent targeting the primary epitopes HNW, HDW, and HHW and secondary epitopes HRW, and HGW could have LWW added as either a corrupting primary or secondary epitope, but HGW could only be added as a corrupting primary epitope, in which case its binding probability would be updated to that of a primary epitope.

### Consistent Over- or Under-Estimation of Affinity Reagent Trimer Binding

Similar to the Missed Trimers During Affinity Reagent Characterization section above, binding of affinity reagents to proteins in the proteome was simulated with surface NSB 0.1% and affinity reagent models were corrupted prior to decoding. In this analysis, epitope binding probabilities were adjusted to be systematically higher or lower than the true values. This models a situation where the affinity reagent characterization method determines the correct trimer epitopes targeted by the affinity reagent, but systematically over or under-estimates the strength of binding (modeled by binding probability). The manipulation entailed applying some fold-change shift to the binding probability of the epitopes such that the primary epitopes of the affinity reagent are shifted by a desired amount. For example, to model a shift of +0.25 for an affinity reagent with true primary epitope binding probability of 0.25, the binding probability of every epitope of the affinity reagent was multiplied by 2. In this case, a primary epitope with true binding probability of 0.25 will be assumed to bind with a probability of 0.5 when performing decoding. Similarly, this same multiplicative shift may be applied to secondary binding epitopes. For example, a secondary epitope with binding probability 0.2 would then have binding probability 0.4. Similarly, adjustments may be made which adjust binding probabilities to be less. In some analyses, the severity of the corruption was modulated by only corrupting a fraction of the affinity reagents. For example, 50% of the affinity reagents may be impacted meaning half of the affinity reagents have a systematic error in their binding probabilities while the rest are not impacted.

### Noisy Affinity Reagent Characterization

Similar to the Missed Trimers During Affinity Reagent Characterization section above, binding of affinity reagents to proteins in the proteome was simulated with surface NSB 0.1% and affinity reagent models were corrupted prior to decoding. In this analysis, random noise was applied to the characterized epitope binding probabilities. The random noise was applied to a random fraction of the affinity reagents in the set. For any affinity reagent impacted by noise, all primary and secondary epitopes were subjected to some degree of noise as well as the affinity reagent non-specific binding rate. The binding probabilities were perturbed according to the method described in the Application of Noise to Affinity Reagent Binding Probabilities section above with the amount of noise ranging between fractional MAD 0 and 0.75.

### Simulation of Cell-Line and Plasma Experiments

### Protein Abundance Database Processing

The protein composition of each sample was modeled using protein abundances downloaded from PaxDb v4.1 (Wang et. al., Molecular Cellular Proteomics, 8:492-500 (2012). doi: 10.1074/mcp.0111.014704, which is incorporated herein by reference). Specifically, plasma protein abundances were from the “H. sapiens—Plasma (Integrated)” dataset (https://pax-db.org/downloads/4.1/datasets/9606/9606-PLASMA-integrated.txt downloaded September, 2021). Cell-line abundances were from the dataset “H. sapiens—Cell line, Hela, SC (Nagaraj,MSB,2011)”(pax-db.org/downloads/4.1/datasets/9606/9606-hela_Nagaraj_2011.txt built from high resolution mass spectrometry analysis of HeLa cells (Nagaraj Molecular Systems Biology, 7:548 (2011). doi:10.1038/msb.2011.81, which is incorporated herein by reference). The identities of proteins in the PaxDb data were mapped to the identities of proteins in the Uniprot human protein sequence database (see the Protein Sequence Databases section above) using the PaxDb to Uniprot mapping available from the PaxDb maintainers available at https://pax-db. org/downloads/4.1/mapping_files/uniprot_mappings/full_uniprot_2_paxdb.04.2015.tsv.zip (downloaded September, 2021). Any proteins present in the PaxDb database that could not be mapped to the UniProt sequence database were removed from the sample. 4,342 of 4,492 entries (97%) in the plasma database were successfully mapped with no unmapped protein comprising more than 1% of the sample. 8,554 of 8,817 entries (97%) in the cell database were successfully mapped with no unmapped protein comprising more than 1% of the sample. In some cases, more than one entry in a PaxDb database mapped to a single UniProt identifier in the sequence database. In these cases, only the first entry was retained. In the plasma database, 99 database entries were dropped as a result of this operation (4,243 entries remained). In the cell-line database, 145 entries were dropped (8,409 entries remained). Neither of these operations dropped any entries comprising more than 1% of the corresponding sample. 25, and 97 proteins with abundance 0 were removed from the plasma and cell-line database, respectively. After filtering, the abundance databases were normalized to sum to 1.

### Imputation of Protein Abundances (Plasma)

Abundances were imputed for proteins in the human protein sequence database not represented in the modeled plasma sample (see Protein Abundance Database Processing section above). This process resulted in a ‘complete’ plasma sample containing 20,235 proteins with 12 orders of magnitude in dynamic range of abundance. The distribution of abundances in the complete plasma sample was modeled as a semi-Gaussian distribution (Eriksson, Nature Biotechnology, 25:651-655 (2007). doi:10.1038/nbt1315, which is incorporated herein by reference):

Let f(a|μ, σ) be the normal distribution probability density function with mean μ and standard deviation σ evaluated at x

$$f ( x \vert μ , σ ) = \frac{1}{\sqrt{2 π {σ}^{2}}} exp ( - \frac{{( x - μ )}^{2}}{2 {σ}^{2}} )$$

Let:
Aₘₐₓ be the highest protein abundance in the modeled plasma sample pre-imputation,
σₚ=1.2
μₚ=log₁₀(Aₘₐₓ)−5σₚ,
[symbol] =log₁₀(Aₘₐₓ)−12
Let g(a) be a function proportional to the probability density of the semi-Gaussian distribution at abundance a. g(a)=
f(log₁₀(a)|μ=μₚ, σ=σₚ) if log₁₀(a)≥μₚ
f(μₚ|μ=μₚ, σ=σₚ) if [symbol] ≤log₁₀(a)<μₚ
0 if log₁₀(a)<

[symbol]

Next, a probability density function for abundances of proteins needing to be imputed was estimated. A threshold was set for ‘high-abundance’ proteins t=Aₘₐₓ−4 on the reasoning that any protein with log₁₀(abundance)>t present in the ‘complete’ plasma sample would be accurately represented in PaxDb (i.e., not impacted by detection bias). The probability density of the PaxDb proteins was estimated by computing a histogram (50 bins) on their log-10 transformed abundances and normalizing the values at each bin such that the total area of the histogram is 1.

A scaling factor α was computed to adjust the high-abundance tail of the complete sample abundance distribution g(x) to match the probability density of protein abundances>t in PaxDb:

$$α = \frac{{\sum }_{j} g ( 1 {0}^{{a}_{j}} ) {d}_{j}}{{\sum }_{j} {g ( 1 {0}^{{a}_{j}} )}^{2}}$$

with
{a₀, a₁, a₂, . . . aⱼ}: the j bin centers of the histogram of log-10 PaxDb abundances with a>t, and
{d₀, d₁, d₂, . . . dⱼ}: the density corresponding to those bin centers.

A kernel density estimate K was fit to the log 10-transformed plasma abundance values using a Gaussian kernel with σ=0.2 and was subtracted from the scaled semi-Gaussian distribution to estimate a function proportional to the density of the probability distribution on abundances for imputed proteins: h(x)=αg(x)−K(x). The function h(x) was evaluated at 500 abundance values spread equally in base-10 logspace between log₁₀(Aₘₐₓ)−12 and log 10 abundance log₁₀(Aₘₐₓ). Any points where h(x) evaluated to less than zero were set to zero. A continuous probability distribution was fit to this lattice of sample points using linear interpolation and then normalized such that the total probability of the distribution was 1. The abundances of the 16,017 proteins in the UniProt database not represented in the processed PaxDb dataset were set to random samples from the aforementioned distribution. The resulting abundances are converted to molar fraction estimates by dividing each abundance by the sum of all abundances.

### Imputation of Protein Abundances (Cell-Line)

Abundances were imputed for proteins in the human protein sequence database not represented in the modeled cell-line sample (see Protein Abundance Database Processing section above). This process resulted in a ‘complete’ cell-line sample containing 20,235 proteins with 10 orders of magnitude in dynamic range of abundance. The “complete” cell-line sample was modeled as an adjusted skewed-normal distribution on log 10-transformed abundances:
g(x)=2.45*skewnorm.pdf(x|α=−2.12, μ=4.5, σ=2.55)
where skewnorm.pdf is the probability density function of the skewed normal distribution.

A kernel density estimate K (Gaussian kernel, σ=0.2) was fit to the log 10-transformed abundances of all entries in the processed PaxDb database for the cell-line sample. The function h(x) was evaluated at 500 abundance values spread equally in base-10 logspace between log 10 abundance log₁₀(Aₘₐₓ)−10 and log 10 abundance log₁₀(Aₘₐₓ). Any points where h(x) evaluated to less than zero were set to zero. A continuous probability distribution was fit to this lattice of sample points using linear interpolation and then normalized such that the total probability of the distribution was 1. The abundances of the 11,923 proteins in the Uniport database not represented in the processed PaxDb dataset were set to random samples from the aforementioned distribution. The resulting abundances are converted to molar fraction estimates by dividing each abundance by the sum of all abundances.

### Depleted Plasma Sample

To model a plasma sample where the most abundant proteins were depleted from the sample (e.g., using a commercially-available affinity column), the abundances of the top-20 most abundant proteins in the imputed plasma sample (see Imputation of Protein Abundances (Plasma) section above) were reduced by 99% and the abundances renormalized to sum to 1 to serve as an estimate of molar fraction.

### Simulating Protein Deposition

Deposition of a sample containing n proteins of abundances {a₁, a₂, a₃, . . . aₙ} on an array was modeled as a multinomial distribution. The protein abundances were normalized to probabilities summing to 1

$${p}_{i} = \frac{{a}_{i}}{{\sum }_{j = 1}^{j = n} {a}_{j}} .$$

To determine the counts of each protein deposited on an array with N addresses, a random sample is made from the multinomial distribution parameterized with the probabilities {p₁, p₂, p₃, . . . pₙ} and N trials.

### Simulation of Binding Data

For each sample type (cell, plasma, depleted plasma), binding was simulated for 5 technical replicate protein arrays. The 300 affinity reagents used for binding targeted the first 300 optimal targets (see Selection and Evaluation of Optimal Affinity Reagent Trimer Targets section above) and used the binding model described in the Biosimilar Affinity Reagent Model section above with a surface non-specific binding rate of 0.001. To simulate random variation in binding replicate-to-replicate, the binding probabilities of affinity reagents were perturbed for each replicate using the method described in the Application of Noise to Affinity Reagent Binding Probabilities section above with fractional mean absolute deviation 0.1. Binding for each flow cell was then simulated as described in the Simulation of Stochastic Affinity Reagent Binding section above.

### Decoding of Binding Data

Protein decoding was performed individually for each replicate as described in the Protein Decoding section above. The human FASTA sequence database (see Protein Sequence Databases section above) was used to define protein candidate sequences. The affinity reagent model used for decoding of all replicates was the original affinity reagent set referenced in the Simulation of Binding Data section above prior to application of random noise. The decoding method assumed a surface non-specific binding rate of 0.001.

### Determining a Probability Threshold for Protein Quantification

At a given identification probability threshold pₜ, proteins in samples may be quantified by counting the number of identifications for that protein in the decoding output with probability p>pₜt. However, if the probability threshold is set too low, many false positive identifications may occur resulting in low quantitative specificity. If the probability threshold is set too high, false negative identifications may occur, resulting in low quantitative sensitivity. For each replicate flow cell analyzed, decoding results were processed with probability thresholds: log(p)=0, −1×10\hat{ }(−20), −1×10\hat{ }(−16), −1×10\hat{ }−14, −1×10\hat{ }−12, −1×10\hat{ }−11, −1×10\hat{ }−10, −1×10\hat{ }-9, −1× 10\hat{ }−8, −1× 10\hat{ }−7, −1×10\hat{ }−6, −1×10\hat{ }−5, −1×10\hat{ }−4, −1×10\hat{ }−3, −1×10\hat{ }−2, −0.1, −0.2, and −0.3.

For each threshold evaluated:
For every unique protein identified at least once in the dataset:
   Compute the number of reported identifications for the protein that were true positive (i.e., correct identifications) and false positives (i.e., spots incorrectly identified as the protein)
   Compute the specificity of quantification for this protein:

$$\frac{\# true positives}{\# identifications}$$

If the protein has specificity <0.9, label it as non-specific identification
Compute the ‘non-specific identification rate’: the fraction of proteins that fall into the “non-specific identification” class

The lowest threshold value resulting in a non-specific identification rate <0.1% for every replicate analyzed was used for downstream quantification analyses.

### Quantitative Statistics

After thresholding by identification probability, the following statistics were computed for each analysis:
Specificity of protein identification was computed as described in the Determining a Probability Threshold for Protein Quantification section above.
Proteins with at least 1 identification in a given replicate were deemed ‘identified’ in that replicate.
Proteome coverage for a replicate was the percentage of proteins identified at least once in the replicate among all proteins present in the sample.
Reproducibility of quantification (CV %) for a protein across replicates was computed using the number of counts for that protein in each replicate:

$$100 \times \frac{s t a ndard deviation of counts}{mean of counts} .$$

Proteins not identified in a replicate were assigned count mean of counts 0.

### Example II

### Use of Markov Chain Monte Carlo Methods to Generate Pseudo Sequences for Semi-Censored Decode

This Example describes a Markov model that is useful for predicting non-binding probability for use in a semi-censored decode method. Advantageously, the Markov model facilitates prediction of non-binding probabilities in a way that accounts for the length of proteins in a given proteome but is agnostic to variability in amino acid sequences for those proteins. The Markov model is used to generate a set of pseudo sequences for each unique protein length L in a proteome of interest. An affinity reagent's non-binding probability can be predicted for each pseudo sequence and the mean or median non-binding prediction of the set of pseudo sequences of length L can be used as the predicted semi-censored non-binding probability for a candidate protein having any amino acid sequence of the same length.

A Markov model can be characterized as a set of finite states, with transition probabilities between these states. These transition probabilities are dependent only on the current state. An example of the model used is described by the transition matrix below. Here, a given row represents a potential current trimer state, and the entries of that row represent the transition probabilities from the rows current state to a state represented by the column label.

$$\begin{matrix} & & & Next & State & & & & \\ & & AAA , & AAC , & AAD , & … , & CYY , & … , & YYY \\ & AAA & 0.1 , & 0.4 , & 0. , & … , & 0. & … , & 0. \\ & AAC & 0. , & 0. , & 0. , & … , & 0. , & … , & 0. \\ Current State & AAD & 0. , & 0. , & 0. , & … , & 0. , & … , & 0. \\ & … & … , & … , & … , & … , & … , & … , & … \\ & CYY & 0. , & 0. , & 0. , & … , & 0. , & … , & 0.2 \\ & … & … , & … , & … , & … , & … , & … , & … \\ & YYY & 0. , & 0. , & 0. , & … , & 0. , & … , & 0.2\end{matrix}$$

With the trimer parameterization of the Markov model, the first two amino acids of any valid next state must maintain the last two amino acids of the current state, therefore, many state transitions are not possible and have a transition probability of 0. As an example, given the current state “AAA” as denoted by row 1, a transition to state “CYY” is impossible, as the last two amino acids of the current state, “AA”, are not maintained as the first two amino acids of the next state. Potentially valid transitions can also have a transition probability of 0 if the training data do not contain such a transition. Purely as an example, the valid transition “AAA” to “AAD” is shown as having a transition probability of 0. A sample can be generated from the Markov model by first stochastically choosing an initial state and history. Further states are then determined by stochastically choosing the next state based on the transition probabilities of the current state. This random walk can be terminated after a pre-determined number of transitions.

For each state, transition probabilities are learned based on observed transitions within the proteome. Sequences generated from this model mimic sequence characteristics of the real proteome (e.g. the amino acid composition). The proteome can be decoded with reference to a first set of candidate proteins that includes native amino acid sequences expected to be present in the proteome. The pseudo sequences are amino acid sequences that are non-native to the proteome. Each pseudo sequence has an amino acid sequence length that is identical to the native sequence that it represents in a set of candidate proteins. If the pseudo proteins were used for uncensored decoding, their average predicted non-binding probability (the uncensored non-binding probability is simply 1−predicted binding probability) approximates the predicted non-binding probability of the “average” sequence—one that represents the amino acid composition of the proteome of interest.

As evident from the above description, non-binding probability can be determined in a way that is strictly length dependent such that variability in amino acid sequences do not affect the calculations. Two proteins of the same length will always have the same non-binding likelihood for a given affinity reagent using these methods.

A similar model can be built based on sequence regions other than trimers. For example, trimers can be replaced in the above model with monomers, dimers, tetramers or pentamers. As the length of the sequence region increases, effectiveness of the model may improve provided adequate training data are available. Shorter lengths such as monomers, dimers and trimers may be preferred for proteomes that are similar in size to the human proteome or smaller.

The Markov model was compared to a binning approach. The binning approach was performed as follows: essentially all the proteins in the human proteome were aggregated into bins of proteins of similar lengths. Within each bin the uncensored non-binding likelihood was predicted for each protein (i.e. (1−P(binding|protein))). The median value was used as the semi-censored non-binding likelihood for the entire bin.

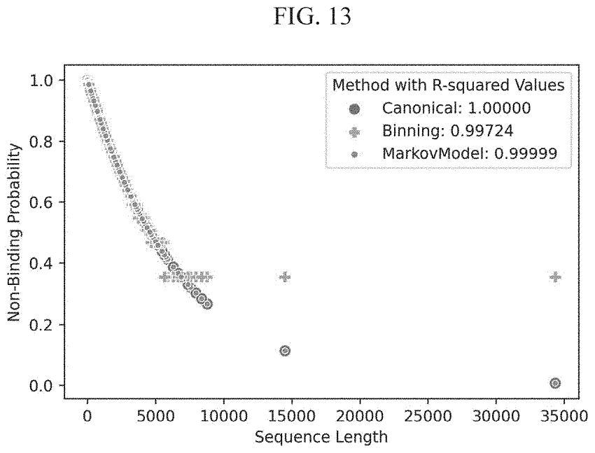

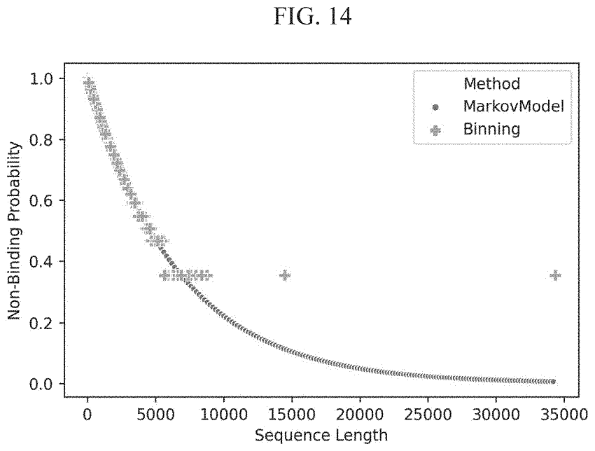

FIG. 13 shows predicted non-binding probabilities by sequence length for different semi-censored decode approaches. The results indicate that the fit of the Markov Model based approach outperforms the binning approach by reducing the R-squared value when compared to the use of a trimer-based probability adjustment. The probability adjustment was determined from

$$θ = {\sum }_{1}^{8000} {p}_{trimer \_ i} * ( 1 - {bp}_{t rimer \_ i} )$$

$$P ( non\_binding \vert L ) = {θ}^{L - 2}$$

Where L is the length of the protein of interest (identified as “canonical” in FIG. 13). FIG. 14 shows non-binding probability predictions for sequences of arbitrary length using different semi-censored decode approaches. The results indicate that pseudo sequences can be used to predict non-binding for sequences of arbitrary length.

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of performing a protein binding assay, comprising:
(a) contacting a plurality of different affinity reagents with a plurality of extant proteins in a sample;
(b) acquiring binding data from step (a), wherein the binding data comprises a plurality of binding profiles, wherein each of the binding profiles comprises a plurality of binding outcomes for binding of an extant protein of step (a) to the plurality of different affinity reagents, wherein individual binding outcomes of the plurality of binding outcomes comprise a measure of binding between an extant protein of step (a) and a different affinity reagent of the plurality of different affinity reagents, each of the binding profiles comprising positive binding outcomes and negative binding outcomes;
(c) providing a database comprising information characterizing or identifying a plurality of candidate proteins;
(d) providing a binding model for each of the different affinity reagents;
(e) determining a probability for each of the affinity reagents binding to each of the candidate proteins in the database according to the binding model, wherein the determining comprises computing probabilities for the positive binding outcomes and for the negative binding outcomes, and wherein the positive binding outcomes are weighted more heavily relative to the negative binding outcomes; and
(f) identifying the extant proteins as selected candidate proteins, the selected candidate proteins being candidate proteins in the database having a probability for binding each of the affinity reagents that is most compatible with the plurality of binding outcomes for the extant proteins.

2. The method of claim 1, further comprising providing a non-specific binding rate comprising a probability of a non-specific binding event occurring for one or more of the different affinity reagents.

3. The method of claim 2, wherein the non-specific binding event comprises binding of the one or more of the different affinity reagents to a solid support attached to the extant protein.

4. The method of claim 1, wherein the computing of the probabilities for the positive binding outcomes comprises determining probability of positive binding events occurring between each candidate protein in the plurality of candidate proteins and each of the affinity reagents.

5. The method of claim 4, wherein the probability of the positive binding event is normalized with respect to the lengths of the candidate proteins.

6. The method of claim 5, wherein the probability of the positive binding event is normalized using a binomial approximation, an exact Poisson binomial or an estimated Poisson binomial.

7. The method of claim 4, wherein the computing of the probabilities for the negative binding outcomes comprises determining probability of a negative binding event occurring between each candidate protein in the plurality of candidate proteins and each of the affinity reagents.

8. The method of claim 7, wherein the probability of the negative binding event is normalized with respect to the lengths of the candidate proteins.

9. The method of claim 8, wherein the probability of the negative binding event is normalized using a binomial approximation, an exact Poisson binomial or an estimated Poisson binomial.

10. The method of claim 4, wherein the computing of the probabilities for the negative binding outcomes comprises determining probability of a negative binding event occurring between each pseudo protein in a plurality pseudo proteins and each of the affinity reagents.

11. The method of claim 10, wherein amino acid sequences in the plurality of pseudo proteins have full-lengths that are identical to the full-lengths for amino acid sequences in the plurality of candidate proteins.

12. The method of claim 11, wherein the plurality of pseudo proteins lacks any full-length amino acid sequences that are present in the plurality of candidate proteins.

13. The method of claim 11, wherein the plurality of pseudo proteins lacks a subset of the full-length amino acid sequences that are present in the plurality of candidate proteins.

14. The method of claim 10, wherein amino acid sequences in the plurality of pseudo proteins are generated by sampling of amino acid sequences in the plurality of candidate proteins using a Markov chain, generative adversarial network or length-based binning.

15. The method of claim 1, further comprising determining the probability that the extant protein identified in step (f) is the selected candidate protein.

16. The method of claim 1, wherein the positive binding outcomes and negative binding outcomes are represented by non-binary values in the binding profile.

17. The method of claim 1, wherein step (e) comprises computing a probability matrix comprising the probabilities of a positive binding outcome for each of the affinity reagents binding to each of the candidate proteins in the database.

18. The method of claim 17, wherein step (e) further comprises computing a probability matrix comprising the probabilities of a negative binding outcome for each of the affinity reagents binding to each of the candidate proteins in the database.

19. A method for identifying an extant protein using a detection system, comprising
(a) acquiring signals from a plurality of binding reactions carried out in a detection system, wherein the binding reactions comprise contacting a plurality of different affinity reagents with a plurality of extant proteins in a sample;
(b) processing the signals in the detection system to produce a plurality of binding profiles, wherein each of the binding profiles comprises a plurality of binding outcomes for binding of an extant protein of step (a) to the plurality of different affinity reagents, wherein individual binding outcomes of the plurality of binding outcomes comprise a measure of binding between an extant protein of step (a) and a different affinity reagent of the plurality of different affinity reagents, each of the binding profiles comprising positive binding outcomes and negative binding outcomes,
(c) providing as inputs to the detection system a database comprising information characterizing or identifying a plurality of candidate proteins;
(d) providing as inputs to the detection system a binding model for each of the different affinity reagents;
(e) processing the plurality of binding profiles in the detection system to determine a probability for each of the affinity reagents binding to each of the candidate proteins in the database according to the binding model; and
(f) outputting from the detection system an identification of selected candidate proteins, the selected candidate proteins being candidate proteins in the database having a probability for binding each of the affinity reagents that is most compatible with the plurality of binding outcomes for the extant proteins.

20. A detection system, comprising:
(a) a detector configured to acquire signals from a plurality of binding reactions occurring between a plurality of different affinity reagents and a plurality of extant proteins in a sample;
(b) a database comprising information characterizing or identifying a plurality of candidate proteins;
(c) a computer processor configured to:
(i) communicate with the database,
(ii) process the signals to produce a plurality of binding profiles,
(ii) wherein each of the binding profiles comprises a plurality of binding outcomes for binding of an extant protein of (a) to the plurality of different affinity reagents, wherein individual binding outcomes of the plurality of binding outcomes comprise a measure of binding between an extant protein of (a) and a different affinity reagent of the plurality of different affinity reagents, each of the binding profiles comprising positive binding outcomes and negative binding outcomes,
(iii) process the binding profiles to determine a probability for each of the affinity reagents binding to each of the candidate proteins in the database according to a binding model for each of the affinity reagents; and
(iv) output an identification of selected candidate proteins, the selected candidate proteins being candidate proteins in the database having a probability for binding each of the affinity reagents that is most compatible with the plurality of binding outcomes for the extant proteins.
